(19) United States
(12) Patent Application Publication
(10) Pub. No.: US 2009/0312321 A1
(43) Pub. Date: Dec. 17, 2009

(54) COMPOSITIONS AND METHODS FOR FGF RECEPTOR KINASES INHIBITORS

(75) Inventors: Pingda Ren, San Diego, CA (US); Guobao Zhang, San Diego, CA (US); Shuli You, Shanghai (CN); Taebo Sim, Seoul (KR); Nathanael S. Gray, Boston, MA (US); Yongping Xie, San Diego, CA (US); Xing Wang, San Diego, CA (US); Yun He, Shanghai (CN)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton, BM (BM)

(21) Appl. No.: 12/300,088

(22) PCT Filed: Apr. 6, 2007
(86) PCT No.: PCT/US07/08699

§ 371 Date: Jul. 24, 2009

Related U.S. Application Data
(60) Provisional application No. 60/747,258, filed on May 15, 2006.

(51) Int. Cl. A61K 31/5377; C07D 471/04; A61K 31/4375; C07D 487/04; A61K 31/519

(52) U.S. Cl. 514/234.5; 546/122; 514/300; 544/127; 544/256; 514/262.1; 514/252.16

(57) ABSTRACT

Described are compounds, pharmaceutical compositions comprising such compounds, and methods of using such compounds to treat or prevent disease or disordered associated with abnormal or deregulated kinase activity, particularly diseases or disorders that involve abnormal activity of kinases such as Abl, ALK, AMPK, Aurora, Axl, Bcr-Abl, BIK, Bmx, BRK, BTK, c-Kit, CSK, cSrc, CDK1, CHK2, CK1, CK2, CaMKII, CaMKIV, DYRK2, EGFR, EphB1, FES, FGFR1, FGFR2, FGFR3, Flt1, Flt3, FMS, Fyn, GSK3β, IGF-1R, IKKα, IKKβ, IR, IRAK4, ITK, JAK2, JAK3, JNK1α1, JNK2α, KDR, Lck, LYN, MAPK1, MAPKAP-K2, MEK1, MET, MKK4, MKK6, MST2, NEK2, NLK, p70S6K, PAK2, PDGFR, PDGFRα, PDK1, Pim-2, Plk3, PKA, PKBα, PKCα, PKCtheta, PKD2, c-Raf, RET, ROCK-I, ROCK-II, Ron, Ros, Rsk1, SAPK2a, SAPK2b, SAPK3, SAPK4, SGK, SIK, Syk, Tie2, TrkB, WNK3, and ZAP-70.

### CROSS-REFERENCE

This application claims the benefit of U.S. provisional application Ser. No. 60/747,258 filed May 15, 2006, which is incorporated by reference in its entirety.

### FIELD OF THE INVENTION

Compounds, methods of making such compounds, pharmaceutical compositions and medicaments comprising such compounds, and methods of using such compounds to treat or prevent diseases or conditions associated abnormal activity of kineases are described.

### BACKGROUND OF THE INVENTION

The protein kinases represent a large family of proteins, which play a central role in the regulation of a wide variety of cellular processes and maintaining control over cellular function. A partial, non-limiting, list of these kinases include: receptor tyrosine kinases such as platelet-derived growth factor receptor kinase (PDGF-R), the receptor kinase for stem cell factor, c-kit, the nerve growth factor receptor, trkB, and the fibroblast growth factor receptor, FGFR3; non-receptor tyrosine kinases such Abl and the fusion kinase BCR-Abl, Fes, Lck and Syk; and serine/threonine kinases such as b-RAF, MAP kinases (e.g., MKK6) and SAPK2□. Aberrant kinase activity has been observed in many disease states including benign and malignant proliferative disorders as well as diseases resulting from inappropriate activation of the immune and nervous systems.

### SUMMARY OF THE INVENTION

Described are compounds, pharmaceutical compositions comprising such compounds and methods of using such compounds to treat or prevent diseases or disorders associated with abnormal or deregulated kinase activity, particularly diseases or disorders that involve abnormal activities of kinases such as Abl, ALK, AMPK, Aurora, Axl, Bcr-Abl, BIK, Bmx, BRK, BTK, c-Kit, CSK, cSrc, CDK1, CHK2, CK1, CK2, CaMKII, CaMKIV, DYRK2, EGFR, EphB1, FES, FGFR1, FGFR2, FGFR3, Flt1, Flt3, FMS, Fyn, GSK3β, IGF-1R, IKKα, IKKβ, IR, IRAK4, ITK, JAK2, JAK3, JNK1α1, JNK2α, KDR, Lck, LYN, MAPK1, MAPKAP-K2, MEK1, MET, MKK4, MKK6, MST2, NEK2, NLK, p70S6K, PAK2, PDGFR, PDGFRα, PDK1, Pim-2, Plk3, PKA, PKBα, PKCα, PKCtheta, PKD2, c-Raf, RET, ROCK-I, ROCK-II, Ron, Ros, Rsk1, SAPK2a, SAPK2b, SAPK3, SAPK4, SGK, SIK, Syk, Tie2, TrkB, WNK3, and ZAP-70.

Described are small molecular compounds which prevent diseases or disorders associated with abnormal or deregulated kinases activity, particularly diseases or disordered that involve abnormal activation of the FGFR kinase.

In one aspect are compounds having the structure of Formula (I):

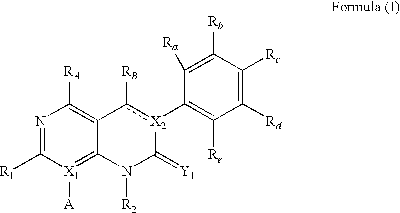

wherein:

each of R₁, R₂, R_{A}, and R_{B} is independently —H, —OH, amino, halogen, —R′, —OR′, —C(O)R′, —C(O)OR′, —S(O)₀₋₂R′, —NR′R″, —NR′″NR′R″, —NHCOR′, aliphatic amine, aromatic amine, —R′″OR′, —R′″C(O)OR′, or —R″′C(O)NR′R″,

where R′ is selected from —H, optionally substituted C₁₋₈ alkyl, optionally substituted C₂₋₈ alkenyl, C₅₋₁₂ aryl-C₀₋₆ alkyl, C₅₋₁₂ heteroaryl-C₀₋₆ alkyl, C₃₋₁₂ cycloalkyl-C₀₋₆ alkyl, and C₃₋₁₂ heterocycloalkyl-C₀₋₆ alkyl; R″ is —H or C₁₋₈ alkyl, or R′ and R″ together with the nitrogen atom to form a C₃₋₁₀ heterocycloalkyl or C₅₋₁₀ heteroaryl; R″′ is a bond, C₁₋₆ alkylene, or arylene;

wherein any aryl, heteroaryl, cycloalkyl, and heterocycloalkyl of R′, R′″, or the combination of R′ and R″, is optionally substituted by one to three radicals independently selected from halo, hydroxy, nitro, cyano, C₁₋₆ alkyl optionally substituted with hydroxy, C₁₋₆ alkoxy, C₂₋₆ alkenyl, halo-substituted-C₁₋₆ alkyl, and halo-substituted-C₁₋₆ alkoxy;

each of X₁ and X₂ is independently C or N;

A is optional, and when present is —H, —OH, amino, —NRₓR_{y}, halogen, or optionally substituted C₁₋₈ alkyl, where Rₓ is selected from —H, C₁₋₈ alkyl, C₂₋₈ alkenyl, C₅₋₁₂ aryl-C₀₋₆ alkyl, C₃₋₁₂ heteroaryl-C₀₋₆ alkyl, C₃₋₁₂ cycloalkyl-C₀₋₆ alkyl, and C₃₋₁₂ heterocycloalkyl-C₀₋₆ alkyl; R_{y} is —H or C₁₋₈ alkyl, or Rₓ, and R_{y} together with the nitrogen atom to form a C₃₋₁₀ heterocycloalkyl or C₅₋₁₀ heteroaryl;

Y₁ is S, O, or NR_{z}, where R_{z} is selected from the group consisting of —H, C₁₋₈ alkyl, C₂₋₈ alkenyl, C₅₋₁₂ aryl-C₀₋₆ alkyl, C₃₋₁₂ heteroaryl-C₀₋₆ alkyl, C₃₋₁₂ cycloalkyl-C₀₋₆ alkyl, C₃₋₁₂ heterocycloalkyl-C₀₋₆ alkyl, and acyl;

each of Rₐ, R_{b}, R_{c}, R_{d}, and Rₑ is independently —H, —OH, amino, halogen, C₁₋₈ alkyl, C₁₋₈ alkoxy, —OCO—C₁₋₈ alkyl, —COR_{f}, —COOR_{f}, —CONR_{f}R_{g}, —N(R_{f})COR_{g}, or —C₁₋₆ alkyl-NR_{f}R_{g},

where each of R_{f} and R_{g} is independently —H, optionally substituted C₁₋₈ alkyl, optionally substituted C₁₋₈ alkoxy, optionally substituted C₂₋₈ alkenyl, optionally substituted C₃₋₁₀ cycloalkyl, or optionally substituted C₃₋₁₀ cycloalkoxy;

provided that at least one of Rₐ, R_{b}, R_{c}, R_{d}, and Rₑ is C₁₋₈ alkoxy and at least one of Rₐ, R_{b}, R_{c}, R_{d}, and Rₑ is —CONR_{f}R_{g}; and a pharmaceutically acceptable salt, pharmaceutically acceptable N-oxide, pharmaceutically active metabolite, pharmaceutically acceptable prodrug, pharmaceutically acceptable solvate thereof.

In a further or alternative embodiment, Y₁ is O or S. In a further or alternative embodiment, X₁ ═X₂═N. In a further or alternative embodiment, X₁ is N and X₂ is C. In a further or alternative embodiment, X₁═X₂═C. When X₁═X₂═C, In a further or alternative embodiment, A is —H, —OH, amino, or optionally substituted C₁₋₈ alkyl.

In a further or alternative embodiment, R₁ is —H, —OH, amino, —R′, —OR′, —NR′R″, —NR″′NR′R″, or —NHCOR′, where R′ is selected from —H, optionally substituted C₁₋₈ alkyl, optionally substituted

C₂₋₈ alkenyl, C₅₋₁₂ aryl-C₀₋₆ alkyl, C₅₋₁₂ heteroaryl-C₀₋₆ alkyl, C₃₋₁₂ cycloalkyl-C₀₋₆ alkyl, and C₃₋₁₂ heterocycloalkyl-C₀₋₆ alkyl; R″ is —H or C₁₋₈ alkyl, or R′ and R″ together with the nitrogen atom to form a C₃₋₁₀ heterocycloalkyl or C₅₋₁₀ heteroaryl; R′″ is a bond, C₁₋₆ alkylene, or arylene.

In a further or alternative embodiment, R₁ is —H, —R′, —OR′, —NHCOR′, aliphatic amine, or aromatic amine, where R′ is selected from the group consisting of —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl. In a further or alternative embodiment, R₁ is selected from the group consisting of

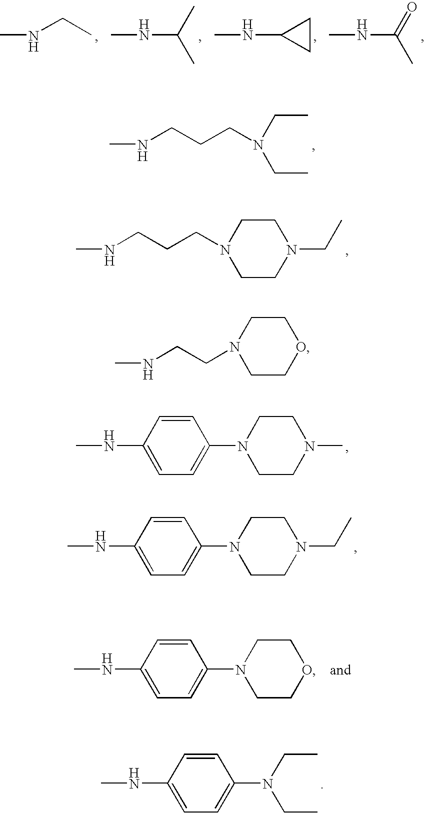

In a further or alternative embodiment, R₂ is —H, —R′, —OR′, —NHCOR′, aliphatic amine, or aromatic amine, where R′ is selected from the group consisting of —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl. In a further or alternative embodiment, R₂ is —R′or —OR′, where R′ is selected from the group consisting of —H.

C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl. In a further or alternative embodiment, R₂ is —H, —OH, C₁₋₆ alkyl, or C₁₋₆ alkoxy. In a further or alternative embodiment, R₂ is —H or C₁₋₆ alkyl.

In a further or alternative embodiment, R_{A} is —H, —R′, —OR′, —NHCOR′, aliphatic amine, or aromatic amine, where R′ is selected from the group consisting of —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl. In a further or alternative embodiment, R_{A} is —H, —OH, C₁₋₆ alkyl, or C₁₋₆ alkoxy. In a further or alternative embodiment, R_{A} is —H.

In a further or alternative embodiment, R_{B} is —H, —R′, —OR′, —NHCOR′, aliphatic amine, or aromatic amine, where R′ is selected from the group consisting of —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl. In a further or alternative embodiment, R_{B} is —H, —OH, C₁₋₆ alkyl, or C₁₋₆ alkoxy. In a further or alternative embodiment, R_{B} is —H.

In a further or alternative embodiment, one of Rₐ, R_{b}, R_{c}, R_{d}, and Rₑ is C₁₋₈ alkoxy and one of Rₐ, R_{b}, R_{c}, R_{d}, and Rₑ is —CONR_{f}R_{g}, where each of R_{f} and R_{g} is independently —H, C₁₋₈ alkyl, C₁₋₈ alkoxy, C₂₋₈ alkenyl, C₃₋₁₀ cycloalkyl, or C₃₋₁₀ cycloalkoxy. In a further or alternative embodiment, one of Rₐ, R_{b}, R_{c}, R_{d}, and Rₑ is selected from the group consisting of

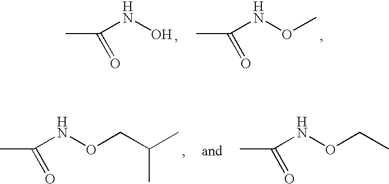

In a further or alternative embodiment, each of Rₐ and Rₑ is independently —H or halogen.

In another aspect are compounds having the structure of Formula (II):

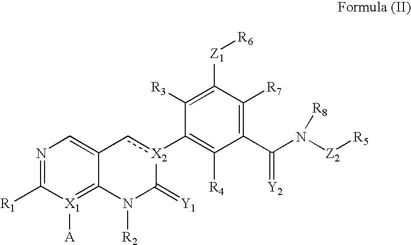

wherein:

each of R₁, and R₂ is independently —H, —OH, amino, halogen, —R′, —OR′, —C(O)R′, —C(O)OR′, —S(O)₀₋₂R′, —NR′R″, —NR″′NR′R″, —NHCOR′, aliphatic amine, aromatic amine, —R″′OR′, —R″′C(O)OR′, or R″′C(O)NR′R″,

where R′ is selected from —H, optionally substituted C₁₋₈ alkyl, optionally substituted C₂₋₈ alkenyl, C₅₋₁₂ aryl-C₀₋₆ alkyl, C₅₋₁₂ heteroaryl-C₀₋₆ alkyl, C₃₋₁₂ cycloalkyl-C₀₋₆ alkyl, and C₃₋₁₂ heterocycloalkyl-C₀₋₆ alkyl; R″ is —H or C₁₋₈ alkyl, or R′ and R″ together with the nitrogen atom to form a C₃₋₁₀ heterocycloalkyl or C₅₋₁₀ heteroaryl; R″′ is a bond, C₁₋₆ alkylene, or arylene;

wherein any aryl, heteroaryl, cycloalkyl, and heterocycloalkyl of R′, R′″, or the combination of R′ and R″, is optionally substituted by one to three radicals independently selected from halo, hydroxy, nitro, cyano, C₁₋₆ alkyl optionally substituted with hydroxy, C₁₋₆ alkoxy, C₂₋₆ alkenyl, halo-substituted-C₁₋₆ alkyl, and halo-substituted-C₁₋₆ alkoxy;

each of X₁ and X₂ is independently C or N;

A is optional, and when present is —H, —OH, amino, —NRₓR_{y}, halogen, or optionally substituted C₁₋₈ alkyl; where R₁ is selected from H, C₁₋₈ alkyl, C₂₋₈ alkenyl, C₅₋₁₂ aryl-C₀₋₆ alkyl, C₃₋₁₂ heteroaryl-C₀₋₆ alkyl, C₃₋₁₂ cycloalkyl-C₀₋₆ alkyl, and C₃₋₁₂ heterocycloalkyl-C₀₋₆ alkyl; R_{y} is —H or C₁₋₈ alkyl, or Rₓ and R_{y} together with the nitrogen atom to form a C₃₋₁₀ heterocycloalkyl or C₅₋₁₀ heteroaryl;

each of Y₁ and Y₂ is independently S, O, or NR_{z}, where R_{z} is selected from the group consisting of

—H, C₁₋₈ alkyl, C₂₋₈ alkenyl, C₅₋₁₂ aryl-C₀₋₆ alkyl, C₃₋₁₂ heteroaryl-C₀₋₆ alkyl, C₃₋₁₂ cycloalkyl-C₀₋₆ alkyl, C₃₋₁₂ heterocycloalkyl-C₀₋₆ alkyl, and acyl;

each of Z₁ and Z₂ is independently S or O;

each of R₃, R₄, and R₇ is independently —H, —OH, amino, halogen, C₁₋₈ alkyl, C₁₋₈ alkoxy, —OCO—C₁₋₈ alkyl, —COR_{f}, —COOR_{f}, —CONR_{f}R_{g}, —N(R_{f})COR_{g}, or —C₁₋₆ alkyl-NR_{f}R_{g},

where each of R_{f} and R_{g} is independently —H, optionally substituted C₁₋₈ alkyl, optionally substituted C₂₋₈ alkenyl, or optionally substituted C₃₋₁₀ cycloalkyl;

each of R₅, R₆, and R₈ is independently —H, —OH, or optionally substituted C₁₋₈ alkyl; and a pharmaceutically acceptable salt, pharmaceutically acceptable N-oxide, pharmaceutically active metabolite, pharmaceutically acceptable prodrug, pharmaceutically acceptable solvate thereof.

In a further or alternative embodiment, Z₁ is O. In a further or alternative embodiment, Z₂ is O. In a further or alternative embodiment, Y₁ is O or S. In a further or alternative embodiment, Y₂ is O or S. In a further or alternative embodiment, X₁═X₂═N. In a further or alternative embodiment, X₁ is N and X₂ is C. In a further or alternative embodiment, X₁═X₂═C. When X₁═X₂═C, in a further or alternative embodiment, A is —H, —OH, amino, or optionally substituted C₁₋₈ alkyl.

In a further or alternative embodiment, R₁ is —H, —OH, amino, —R′, —OR′, —NR′R″, —NR′″NR′R″, or —NHCOR′, where R′ is selected from —H, optionally substituted C₁₋₈ alkyl, optionally substituted C₂₋₈ alkenyl, C₅₋₁₂ aryl-C₀₋₆ alkyl, C₅₋₁₂ heteroaryl-C₀₋₆ alkyl, C₃₋₁₂ cycloalkyl-C₀₋₆ alkyl, and C₃₋₁₂ heterocycloalkyl-C₀₋₆ alkyl; R″ is —H or C₁₋₈ alkyl, or R′ and R″ together with the nitrogen atom to form a C₃₋₁₀ heterocycloalkyl or C₅₋₁₀ heteroaryl; R″ is —H or C₁₋₈ alkyl, or R′ and R″ together with the nitrogen atom to form a C₃₋₁₀ heterocycloalkyl or C₅₋₁₀ heteroaryl; R′″ is a bond, C₁₋₆ alkylene, or arylene.

In a further or alternative embodiment, R₁ is —H, —R′, —OR′, —NHCOR′, aliphatic amine, or aromatic amine, where R′ is selected from the group consisting of —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl. In a further or alternative embodiment, R₁ is selected from the group consisting of

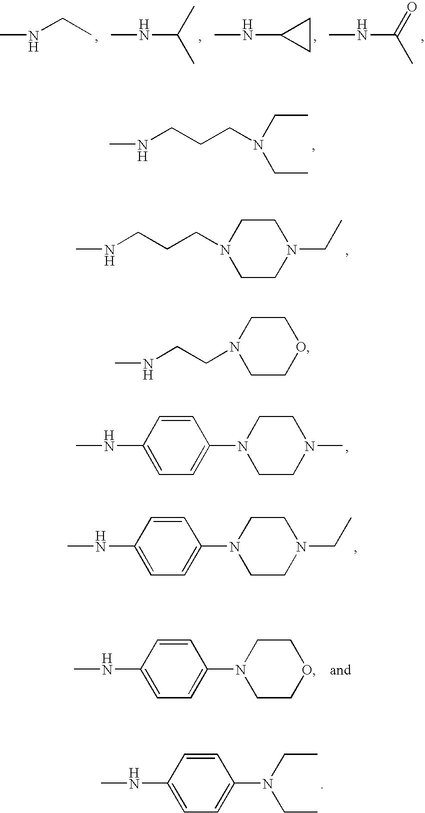

In a further or alternative embodiment, R₂ is —H, —R′, —OR′, —NHCOR′, aliphatic amine, or aromatic amine, where R′ is selected from the group consisting of —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl. In a further or alternative embodiment, R₂ is —R′ or —OR′, where R′ is selected from the group consisting of —H,

C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl. In a further or alternative embodiment, R₂ is —H, —OH, C₁₋₆ alkyl, or C₁₋₆ alkoxy. In a further or alternative embodiment, R₂ is —H or C₁₋₆ alkyl.

In a further or alternative embodiment, R₃ is —H, —OH, halogen, C₁₋₈ alkyl, or C₁₋₈ alkoxy. In a further or alternative embodiment, R₃ is —H. In a further or alternative embodiment, R₄ is —H, —OH, halogen, C₁₋₈ alkyl, or C₁₋₈ alkoxy. In a further or alternative embodiment, R₄ is —H. In a further or alternative embodiment, R₅ is —H or C₁₋₈ alkyl. In a further or alternative embodiment, R₆ is —H or C₁₋₈ alkyl. In a further or alternative embodiment, R₇ is —H, —OH, halogen, C₁₋₈ alkyl or C₁₋₈ alkoxy. In a further or alternative embodiment, R₇ is —H. In a further or alternative embodiment, R₈ is —H or C₁₋₈ clkyl. In a further or alternative embodiment, each of R₃ and R₄ is independently —H or halogen.

In another aspect are compounds having the structure of Formula (III):

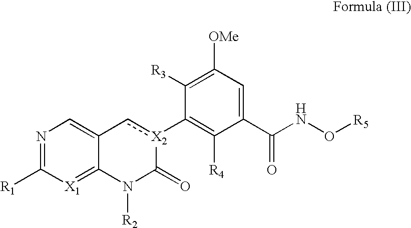

wherein:

R₁ is —H, —R′, —OR′, —NR′R″, —NR′″NR′R″, —NHCOR′, aliphatic amine, or aromatic amine,

where R′ is selected from —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl; R″ is —H or C₁₋₈ alkyl, or R′ and R″ together with the nitrogen atom to form a C₃₋₁₀ heterocycloalkyl or C₅₋₁₀ heteroaryl; R″′ is a bond, C₁₋₆ alkylene, or arylene;

wherein any aryl, heteroaryl, cycloalkyl, and heterocycloalkyl of R′, R′″, or the combination of R′ and R″, is optionally substituted by one to three radicals independently selected from halo, hydroxy, nitro, cyano, C₁₋₆ alkyl optionally substituted with hydroxy, C₁₋₆ alkoxy, C₂₋₆ alkenyl, halo-substituted-C₁₋₆ alkyl, and halo-substituted-C₁₋₆ alkoxy;

R₂ is —H, —OH, halogen, optionally substituted C₁₋₆ alkyl, or optionally substituted C₁₋₆ alkoxy;

each of X₁ and X₂ is independently C or N;

each of R₃ and R₄ is independently —H, —CH₃, halogen, or alkoxyl;

R₅ is —H or optionally substituted C₁₋₆ alkyl; and a pharmaceutically acceptable salt, pharmaceutically acceptable N-oxide, pharmaceutically active metabolite, pharmaceutically acceptable prodrug, pharmaceutically acceptable solvate thereof.

In a further or alternative embodiment, wherein X₁═X₂═N. In a further or alternative embodiment, X₁ is N and X₂ is C. In a further or alternative embodiment, X₁ is CH and X₂═C.

In a further or alternative embodiment, R₁ is —H, —R′, —OR′, —NR′R″, —NR′″NR′R″, or —NHCOR′, where R′ is selected from —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl; R″ is —H or C₁₋₈ alkyl, or R′ and R″ together with the nitrogen atom to form a C₃₋₁₀ heterocycloalkyl or C₅₋₁₀ heteroaryl; R″′ is a bond, C₁₋₆ alkylene, or arylene.

In a further or alternative embodiment, R₁ is —H, —R′, —OR′, —NHCOR′, aliphatic amine, or aromatic amine, where R′ is selected from —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl. In a further or alternative embodiment, R₁ is selected from the group consisting of

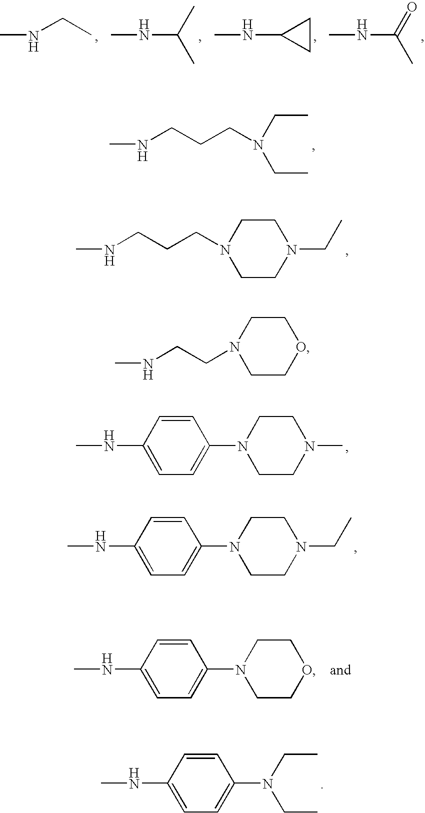

In a further or alternative embodiment, R₂ is —H or C₁₋₆ alkyl. In a further or alternative embodiment, R₃ is —H or CH₃. In a further or alternative embodiment, R₄ is —H or —CH₃. In a further or alternative embodiment, R₅ is —H or C₁₋₆ alkyl. In a further or alternative embodiment, each of R₃ and R₄ is independently —H or halogen.

In a further or alternative embodiment, the compound is selected from the group consisting of:

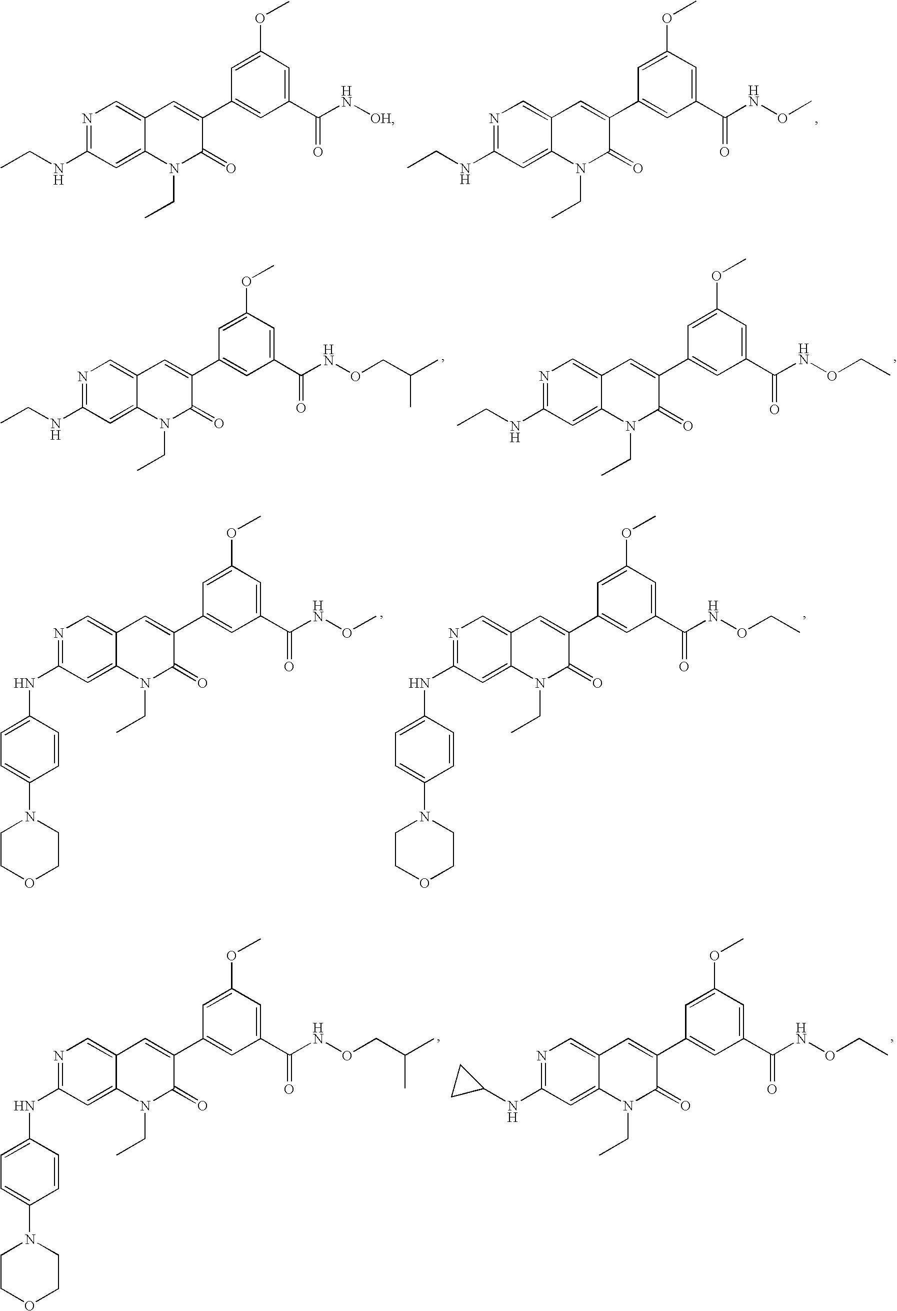

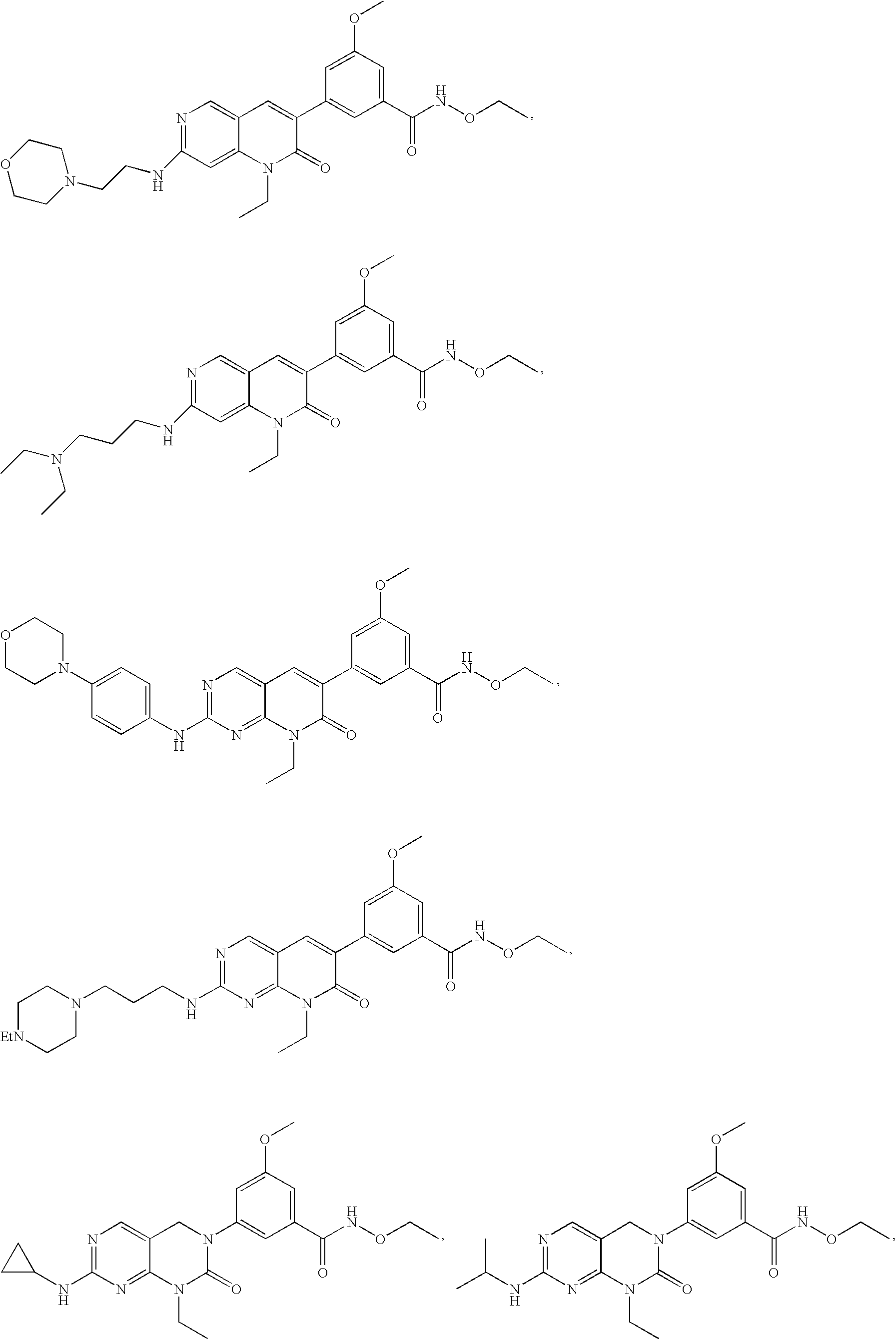

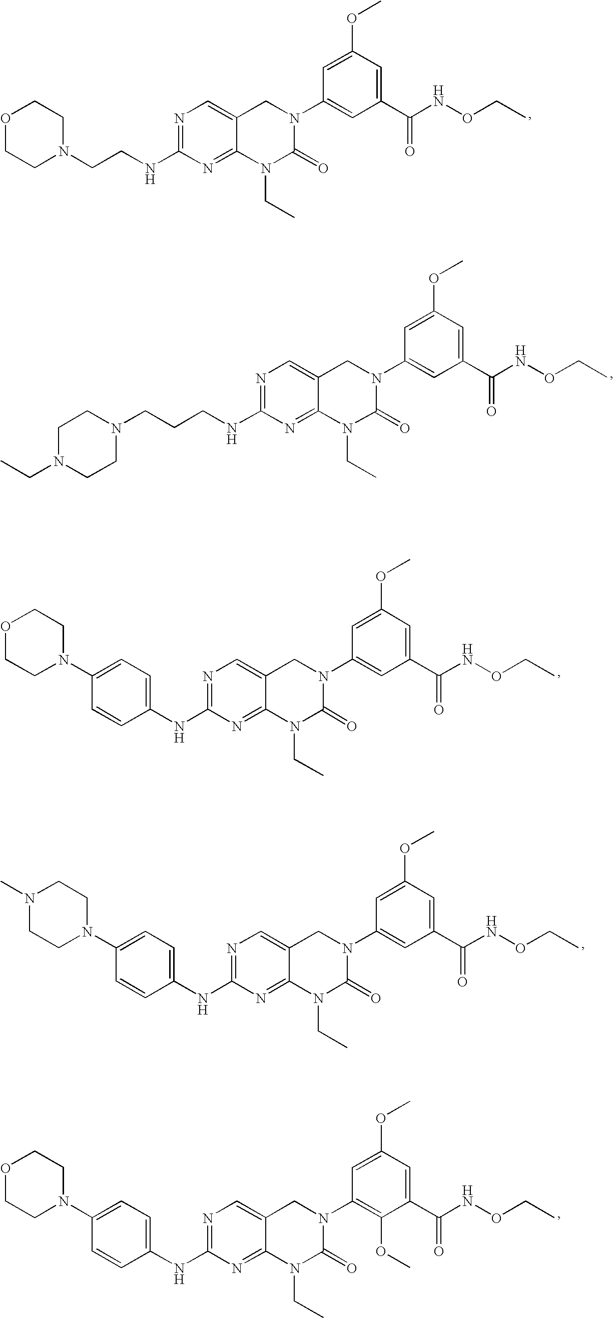

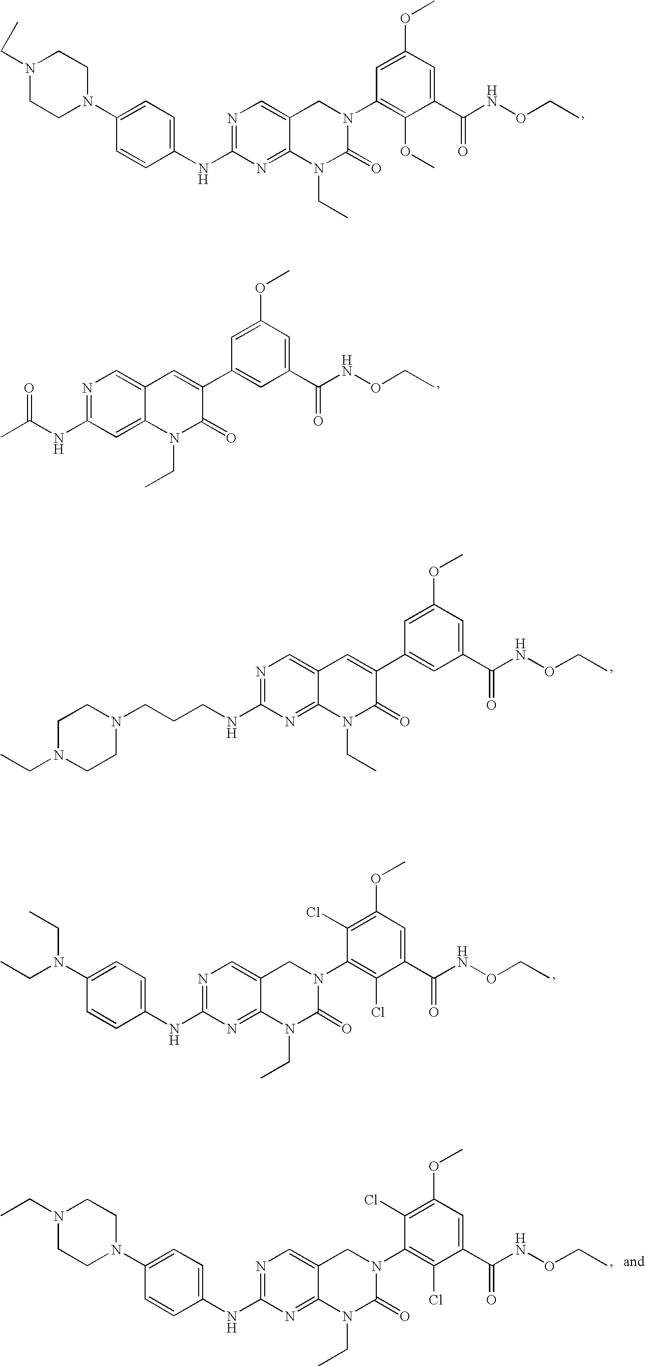

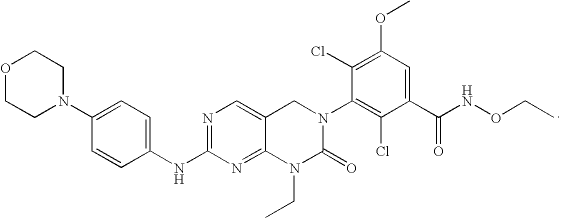

In another aspect are pharmaceutical compositions comprising a therapeutically effective amount of at least one compound of Formula (I), (II), or (III), their respective N-oxide or other pharmaceutically acceptable derivatives, or individual isomers and mixtures of isomers thereof, in admixture with at least one pharmaceutically acceptable excipient.

In another aspect are methods for treating a disease in an animal in which inhibition of kinase activity can prevent, inhibit or ameliorate the pathology and/or symptomology of the disease, which method comprises administering to the animal a therapeutically effective amount of at least one compound of Formula (I), (II), or (III), their respective N-oxide or other pharmaceutically acceptable derivatives, or individual isomers and mixtures of isomers thereof.

In a further or alternative embodiment, the kinase is selected from the group consisting of Abl, ALK, AMPK, Aurora, Axl, Bcr-Abl, BIK, Bmx, BRK, BTK, c-Kit, CSK, cSrc, CDK1, CHK2, CK1, CK2, CaMKII, CaMKIV, DYRK2, EGFR, EphB1, FES, FGFR1, FGFR2, FGFR3, Flt1, Flt3, FMS, Fyn, GSK3β, IGF-1R, IKKα, IKKβ, IR, IRAK4, ITK, JAK2, JAK3, JNK1α1, JNK2α, KDR, Lck, LYN, MAPK1, MAPKAP-K2, MEK1, MET, MKK4, MKK6, MST2, NEK2, NLK, p70S6K, PAK2, PDGFR, PDGFRα, PDK1, Pim-2, Plk3, PKA, PKBα, PKCα, PKCtheta, PKD2, c-Raf, RET, ROCK-I, ROCK-II, Ron, Ros, Rsk1, SAPK2a, SAPK2b, SAPK3, SAPK4, SGK, SIK, Syk, Tie2, TrkB, WNK3, and ZAP-70. In a further or alternative embodiment, the kinase is selected from the group consisting of Abl, BCR-Abl, Bmx, c-Raf, Csk, Fes, FGFR, Flt3, Ikk, IR, JNK, Lck, Mkk, PKC, PKD, Rsk, SAPK, Syk, Trk, BTK, Src, EGFR, IGF, Mek, Ros and Tie2.

In another aspect is the use of a compound of Formula (I), (II), or (III), in the manufacture of a medicament for treating a disease in an animal in which kinase activity contributes to the pathology and/or symptomology of the disease.

In a further or alternative embodiment, the kinase is selected from the group consisting of Abl, ALK, AMPK, Aurora, Axl, Bcr-Abl, BIK, Bmx, BRK, BTK, c-Kit, CSK, cSrc, CDK1, CHK2, CK1, CK2, CaMKII, CaMKIV, DYRK2, EGFR, EphB1, FES, FGFR1, FGFR2, FGFR3, Flt1, Flt3, FMS, Fyn, GSK3β, IGF-1R, IKKα, IKKβ, IR, IRAK4, ITK, JAK2, JAK3, JNK1α1, JNK2α, KDR, Lck, LYN, MAPK1, MAPKAP-K2, MEK1, MET, MKK4, MKK6, MST2, NEK2, NLK, p70S6K, PAK2, PDGFR, PDGFRα, PDK1, Pim-2, Plk3, PKA, PKBα, PKCα, PKCtheta, PKD2, c-Raf, RET, ROCK-I, ROCK-II, Ron, Ros, Rsk1, SAPK2a, SAPK2b, SAPK3, SAPK4, SGK, SIK, Syk, Tie2, TrkB, WNK3, and ZAP-70. In a further or alternative embodiment, the kinase is selected from the group consisting of Abl, BCR-Abl, Bmx, c-Raf, Csk, Fes, FGFR, Flt3, Ikk, IR, JNK, Lck, Mkk, PKC, PKD, Rsk, SAPK, Syk, Trk, BTK, Src, EGFR, IGF, Mek, Ros and/or Tie2.

In a further or alternative embodiment, the disease is selected from the group consisting of chronic myeloid leukemia (CML), acute lymphocytic leukemia, reimplantation of purified bone marrow cells, atherosclerosis, thrombosis, gliomas, sarcomas, prostate cancer, colon cancer, breast cancer, and ovary cancer, small cell lung cancer, psoriasis, scleroderma, fibrosis, protection of stem cells after treatment of chemotherapeutic agents, asthma, allogenic transplantation, tissue rejection, obliterative bronchiolitis (OB), restenosis, Wilms tumors, neuroblastomas, mammary epithelial cancer cells, thanatophoric dysplasia, growth arrest, abnormal bone development, myeloma-type cancers, hypertension, diabetic retinopathy, psoriasis, Kaposi's sarcoma, chronic neovascularization due to macular degeneration, rheumatoid arthritis, infantile haemangioma, rheumatoid arthritis, other autoimmune diseases, thrombin-induced platelet aggregation, immunodeficiency disorders, allergies, osteoporosis, osteoarthritis, neurodegenerative diseases, hepatic ischemia, myocardial infarction, congestive heart failure, other heart diseases, HTLV-1 mediated tumorigenesis, hyperplasia, pulmonary fibrosis, angiogenesis, stenosis, endotoxin shock, glomerular nephritis, genotoxic insults, chronic inflammation, and other inflammatory diseases.

In another aspect are processes for preparing a compound corresponding to Formula (I), (II), or (III), their respective N-oxide or other pharmaceutically acceptable derivatives such as prodrug derivatives, or individual isomers and mixtures of isomers thereof.

### INCORPORATION BY REFERENCE

Unless stated otherwise, all publications and patent applications mentioned in this specification are herein incorporated by reference to the same extent as if each individual publication or patent application is specifically and individually indicated to be incorporated by reference.

### DETAILED DESCRIPTION OF THE INVENTION

The fusion protein BCR-Abl is a result of a reciprocal translocation that fuses the Abl proto-oncogene with the Bcr gene. BCR-Abl is then capable of transforming B-cells through the increase of mitogenic activity. This increase results in a reduction of sensitivity to apoptosis, as well as altering the adhesion and homing of CML progenitor cells. Described are compounds, compositions and methods for the treatment of diseases related to abnormal activities of kinases, particularly Abl, ALK, AMPK, Aurora, Axl, Bcr-Abl, BIK, Bmx, BRK, BTK, c-Kit, CSK, cSrc, CDK1, CHK2, CK1, CK2, CaMKII, CaMKIV, DYRK2, EGFR, EphB1, FES, FGFR1, FGFR2, FGFR3, Flt1, Flt3, FMS, Fyn, GSK3β, IGF-1R, IKKα, IKKβ, IR, IRAK4, ITK, JAK2, JAK3, JNK1α1, JNK2α, KDR, Lck, LYN, MAPK1, MAPKAP-K2, MEK1, MET, MKK4, MKK6, MST2, NEK2, NLK, p70S6K, PAK2, PDGFR, PDGFRα, PDK1, Pim-2, Plk3, PKA, PKBα, PKCα, PKCtheta, PKD2, c-Raf, RET, ROCK-I, ROCK-II, Ron, Ros, Rsk1, SAPK2a, SAPK2b, SAPK3, SAPK4, SGK, SIK, Syk, Tie2, TrkB, WNK3, and ZAP-70. For example, leukemia and other proliferation disorders related to BCR-Abl can be treated through the inhibition of wild type and mutant forms of Bcr-Abl.

### Certain Chemical Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed.

The term “alkenyl group”, as used herein, refers to a hydrocarbon chain having one or more double bonds therein. The double bond of an alkenyl group can be unconjugated or conjugated to another unsaturated group. Suitable alkenyl groups include, but are not limited to, (C₂-C₈)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. The alkenyl moiety may be branched, straight chain, or cyclic (in which case, it would also be known as a “cycloalkenyl” group), and can be unsubstituted or substituted.

The term “alkoxy” as used herein, includes —O-(alkyl), where alkyl is as defined herein. By way of example only, C₁₋₆ alkoxy includes, but is not limited to, methoxy, ethoxy, and the like. An alkoxy group can be unsubstituted or substituted.

The term “alkyl”, as used herein, refers to a hydrocarbon group having from 1 to 10 carbon atoms and can include straight, branched, cyclic, saturated and/or unsaturated features. Whenever it appears herein, a numerical range such as “1 to 10” refers to each integer in the given range; e.g., “1 to 10 carbon atoms” or “C₁₋₁₀” or “(C₁-C₁₀)” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. The alkyl moiety may be a “saturated alkyl” group, which means that it does not contain any alkene or alkyne moieties. Representative saturated alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, sec-butyl, t-butyl, n-pentyl, isopentyl, neopentyl, and n-hexyl, and longer alkyl groups, such as heptyl, and octyl. The alkyl moiety may also be an “unsaturated alkyl” moiety, which means that it contains at least one alkene or alkyne moiety. An “alkene” moiety refers to a group consisting of at least two carbon atoms and at least one carbon-carbon double bond, and an “alkyne” moiety refers to a group consisting of at least two carbon atoms and at least one carbon-carbon triple bond. Representative unsaturated alkyl groups include, but are not limited to, ethenyl, propenyl, butenyl and the like. An alkyl group can be unsubstituted or substituted. Substituted alkyl groups include, but are not limited to, halogen-substituted alkyl groups, such as, by way of example only, trifluoromethyl, pentafluoroethyl, and the like.

The term “alkylamine”, as used herein, refers to the —N(alkyl)ₓH_{y} group, where x and y are selected from the group x=1, y=1 and x=2, y=0. When x=2, the alkyl groups, taken together, can optionally form a cyclic ring system and further when x=2, the alkyl groups can be the same or different. An alkylamine group can be unsubstituted or substituted.

The term “alkynyl” group, as used herein, refers to a hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. The alkynyl moiety may be branched or straight chain, and can be unsubstituted or substituted.

The term “amide”, as used herein, refers to a chemical moiety with formula —C(O)NHR or —NHC(O)R, where R is selected from the group consisting of alkyl, cycloalkyl, aryl, and heterocyclic (bonded through a ring carbon). Amides can be formed from any amine or carboxyl side chain on the compounds described herein. The procedures and specific groups to make such amides are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety. An amide group can be unsubstituted or substituted.

The term “aromatic” or “aryl”, as used herein, refers to a closed ring structure which has at least one ring having a conjugated pi electron system and includes both carbocyclic aryl and heterocyclic aryl (or “heteroaryl” or “heteroaromatic”) groups. The carbocyclic or heterocyclic aromatic group may contain from 5 to 20 ring atoms. The term includes monocyclic or fused-ring polycyclic (i.e., rings which share adjacent pairs of carbon atoms) groups. An aromatic group can be unsubstituted or substituted.

The term “aryloxy”, as used herein, includes —O-aryl group, wherein aryl is as defined herein. An aryloxy group can be unsubstituted or substituted.

The term “bond” or “single bond”, as used herein, refers to a covalent bond between two atoms, either of which may be part of a larger moiety.

The terms “carbocyclic” or “cycloalkyl”, as used herein, refer to a compound which contains one or more covalently closed ring structures, and that the atoms forming the backbone of the ring are all carbon atoms. Such a group may have from 3 to 20 ring carbon atoms and be saturated, partially unsaturated, or fully unsaturated monocyclic, fused bicyclic, spirocyclic, bridged polycyclic or polycyclic ring comprising carbon and hydrogen atoms. Carbocyclic alkyl groups include, but are not limited to, cyclopropyl, cyclobutyl, cyclopentyl, cyclohexyl, cycloheptyl, and cyclooctyl. A carbocyclic aromatic group includes, but is not limited to, phenyl, tolyl, anthracenyl, fluorenyl, indenyl, azulenyl, and naphthyl, as well as benzo-fused carbocyclic moieties such as, by way of example only, dibenzosuberenone, and dibenzosuberone. A carbocyclic group can be unsubstituted or substituted.

The term “ester”, as used herein, refers to a chemical moiety with formula —COOR, where R is selected from the group consisting of alkyl, cycloalkyl, aryl, and heterocyclic (bonded through a ring carbon). Any hydroxy or carboxyl side chain on the compounds described herein can be esterified. The procedures and specific groups to make such esters are known to those of skill in the art and can readily be found in reference sources such as Greene and Wuts, Protective Groups in Organic Synthesis, 3^{rd} Ed., John Wiley & Sons, New York, N.Y., 1999, which is incorporated herein by reference in its entirety. An ester group can be unsubstituted or substituted.

The terms “heteroalkyl” “heteroalkenyl” and “heteroalkynyl”, as used herein, include optionally substituted alkyl, alkenyl and alkynyl moieties and which have one or more skeletal chain atoms selected from an atom other than carbon, e.g., oxygen, nitrogen, sulfur, phosphorus or combinations thereof. A “heteroalkyl” “heteroalkenyl” and “heteroalkynyl” group can be unsubstituted or substituted.

The terms “heteroaryl” or, alternatively, “heteroaromatic”, as used herein, refers to an aryl group that includes one or more ring heteroatoms selected from nitrogen, oxygen, sulfur. By way of example, an N-containing “heteroaromatic” or “heteroaryl” moiety refers to an aromatic group in which at least one of the skeletal atoms of the ring is a nitrogen atom. A polycyclic heteroaryl group may be fused or non-fused. A heteroaryl group can be unsubstituted or substituted.

The term “heterocyclic”, as used herein, refers to ring structures in which the ring backbone contains at least one atom selected from nitrogen, oxygen, and sulfur. Examples of heterocyclic aromatic groups include, but are not limited to, acridinyl, benzo[1,3]dioxole, benzimidazolyl, benzindazolyl, benzoisooxazolyl, benzokisazolyl, benzofuranyl, benzofurazanyl, benzopyranyl, benzothiazolyl, benzo[b]thienyl, benzothiophenyl, benzothiopyranyl, benzotriazolyl, benzoxazolyl, carbazolyl, carbolinyl, cinnolinyl, furanyl, furazanyl, furopyridinyl, furyl, imidazolyl, indazolyl, indolyl, indolidinyl, indolizinyl, isobenzofuranyl, isoindolyl, isoxazolyl, isoquinolinyl, isothiazolyl, naphthylidinyl, naphthyridinyl, oxadiazolyl, oxazolyl, phenoxazinyl, phenothiazinyl, phenazinyl, phenoxathiynyl, thianthrenyl, phenathridinyl, phenathrolinyl, phthalazinyl, pteridinyl, purinyl, puteridinyl, pyrazyl, pyrazolyl, pyridyl, pyridinyl, pyridazinyl, pyrazinyl, pyrimidinyl, pyrimidyl, pyrrolyl, quinazolinyl, quinolinyl, quinoxalinyl, tetrazolyl, thiadiazolyl, thiazolyl, thienyl, triazinyl, (1,2,3,)- and (1,2,4)-triazolyl and the like. In addition, a heterocyclic group can be unsubstituted or substituted. Examples of non-aromatic heterocyclic groups include, but are not limited to, are azepinyl, azepan-2-onyl, azetidinyl, diazepinyl, dihydrofuranyl, dihydropyranyl, dihydrothienyl, dioxanyl, dioxolanyl, 1,4-dioxa-8-aza-spiro[4.5]dec-8-yl, dithianyl, dithiolanyl, homopiperidinyl, imidazolinyl, imidazolidinyl, indolinyl, indolyl, morpholinyl, oxazepinyl, oxepanyl, oxetanyl, oxylanyl, piperidino, piperidyl, piperidinonyl, piperazinyl, pyranyl, pyrazolinyl, pyrazolidinyl, pyrrolidinyl, pyrrolidinonyl, pyrrolinyl, quinolizinyl, thietanyl, tetrahydrofuranyl, tetrahydroquinolyl, tetrahydrothienyl, tetrahydrothiopyranyl, tetrahydropyridinyl, tetrahydropyranyl, thiazepinyl, thiepanyl, thiomorpholinyl, thioranyl, thioxanyl and the like. The heterocyclic group may be fused or non-fused. The terms referring to the groups also encompass all possible tautomers.

The term “halogen”, as used herein, means fluoro, chloro, bromo or iodo. Preferred halogen groups are fluoro, chloro and bromo.

The terms “halo alkyl,” “halo alkenyl,” “halo alkynyl” and “halo alkoxy” include alkyl, alkenyl, alkynyl and alkoxy structures that are substituted with one or more halogen groups or with combinations thereof.

The term “membered ring”, as used herein, can embrace any cyclic structure. The term “membered” is meant to denote the number of skeletal atoms that constitute the ring. Thus, for example, cyclohexyl, pyridine, pyran and thiopyran are 6-membered rings and cyclopentyl, pyrrole, furan, and thiophene are 5-membered rings.

The term “moiety”, as used herein, refers to a specific segment or functional group of a molecule. Chemical moieties are often recognized chemical entities embedded in or appended to a molecule.

The term “protecting group”, as used herein, refers to a chemical moiety which blocks some or all reactive moieties and prevent such groups from participating in chemical reactions until the protective group is removed.

The term “reactant”, as used herein, refers to a nucleophile or electrophile used to create covalent linkages.

The term “sulfonyl” refers to the presence of a sulfur atom, which is optionally linked to another moiety such as an alkyl group, an aryl group, or a heterocyclic group. Aryl or alkyl sulfonyl moieties have the formula —SO₂R′, wherein R′ is alkyl or aryl as defined herein, and include, but are not limited to, methylsulfonyl, ethylsulfonyl and phenylsulfonyl groups. A sulfonyl group can be unsubstituted or substituted. A phenylsulfonyl is optionally substituted with 1 to 3 substituents independently selected from halogen, alkyl, and alkoxy.

Unless otherwise indicated, when a substituent is deemed to be “optionally substituted,” it is meant that the substituent is a group that may be substituted with one or more group(s) individually and independently selected from, for example, alkenyl, alkyl, alkoxy, alkylamine, alkylthio, alkynyl, amide, amino, including mono- and di-substituted amino groups, aryl, aryloxy, arylthio, carbonyl, carbocyclic, cyano, cycloalkyl, halogen, heteroalkyl, heteroalkenyl, heteroalkynyl, heteroaryl, heterocyclic, hydroxy, isocyanato, isothiocyanato, mercapto, nitro, O-carbamyl, N-carbamyl, O-thiocarbamyl, N-thiocarbamyl, C-amido, N-amido, S-sulfonamido, N-sulfonamido, C-carboxy, O-carboxy, perhaloalkyl, perfluoroalkyl, silyl, sulfonyl, thiocarbonyl, thiocyanato, trihalomethanesulfonyl, and the protected compounds thereof. The protecting groups that may form the protected compounds of the above substituents are known to those of skill in the art and may be found in references such as Greene and Wuts, Protective Groups in Organic Synthesis, 3^{rd} Ed., John Wiley & Sons, New York, N.Y., 1999, and Kocienski, Protective Groups, Thieme Verlag, New York, N.Y., 1994, which are incorporated herein by reference in their entirety.

### Certain Pharmaceutical Terminology

The term “acceptable” with respect to a formulation, composition or ingredient, as used herein, means having no persistent detrimental effect on the general health of the subject being treated.

The term “agonist”, as used herein, refers to a molecule such as a compound, a drug, an enzyme activator or a hormone modulator which enhances the activity of another molecule or the activity of a receptor site.

The term “antagonist”, as used herein, refers to a molecule such as a compound, a drug, an enzyme inhibitor, or a hormone modulator, which diminishes, or prevents the action of another molecule or the activity of a receptor site.

The term “carrier”, as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount”, as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The terms “enhance” or “enhancing”, as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

The term “metabolite”, as used herein, refers to a derivative of a compound which is formed when the compound is metabolized.

The term “active metabolite”, as used herein, refers to a biologically active derivative of a compound that is formed when the compound is metabolized.

The term “metabolized”, as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

The term “modulate”, as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator”, as used herein, refers to a molecule that interacts with a target either directly or indirectly. The interactions include, but are not limited to, the interactions of an agonist and an antagonist.

By “pharmaceutically acceptable”, as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The phrase “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

The term “pharmaceutically acceptable salt” of a compound, as used herein, refers to a salt that is pharmaceutically acceptable.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (I), (II), or (III), and a co-agent, are both administered to a patient simultaneously in the form of a single entity or dosage. The term “non-fixed combination” means that the active ingredients, e.g. a compound of Formula (I), (II), or (III), and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical composition”, as used herein, refers to a mixture of an active compound with other chemical components, such as carriers, stabilizers, diluents, dispersing agents, suspending agents, thickening agents, and/or excipients.

A “prodrug”, as used herein, refers to a drug or compound in which metabolic processes within the body converts the drug or compound into a pharmacological active form.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment”, as used herein, include at least partially alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

The term “bioavailability,” as used herein, refers to the rate and extent to which a substance or its active moiety is delivered from a pharmaceutical dosage form and becomes available at the site of action or in the general circulation. Increases in bioavailability refers to increasing the rate and extent a substance or its active moiety is delivered from a pharmaceutical dosage form and becomes available at the site of action or in the general circulation. By way of example, an increase in bioavailability may be indicated as an increase in concentration of the substance or its active moiety in the blood when compared to other substances or active moieties.

### Pharmacology and Utility

Compounds modulate the activity of protein tyrosine kinases and, as such, are useful for treating diseases or disorders in which protein tyrosine kinases, particularly Abl, ALK, AMPK, Aurora, Axl, Bcr-Abl, BIK, Bmx, BRK, BTK, c-Kit, CSK, cSrc, CDK1, CHK2, CK1, CK2, CaMKII, CaMKIV, DYRK2, EGFR, EphB1, FES, FGFR1, FGFR2, FGFR3, Flt1, Flt3, FMS, Fyn, GSK3β, IGF-1R, IKKα, IKKβ, IR, IRAK4, ITK, JAK2, JAK3, JNK1α1, JNK2α, KDR, Lck, LYN, MAPK1, MAPKAP-K2, MEK1, MET, MKK4, MKK6, MST2, NEK2, NLK, p70S6K, PAK2, PDGFR, PDGFRα, PDK1, Pim-2, Plk3, PKA, PKBα, PKCα, PKCtheta, PKD2, c-Raf, RET, ROCK-I, ROCK-II, Ron, Ros, Rsk1, SAPK2a, SAPK2b, SAPK3, SAPK4, SGK, SIK, Syk, Tie2, TrkB, WNK3, and ZAP-70 kinases, contribute to the pathology and/or symptomology of the diseases.

Abelson tyrosine kinase (i.e. Abl, c-Abl) is involved in the regulation of the cell cycle, in the cellular response to genotoxic stress, and in the transmission of information about the cellular environment through integrin signaling. Overall, it appears that the Abl protein serves a complex role as a cellular module that integrates signals from various extracellular and intracellular sources and that influences decisions in regard to cell cycle and apoptosis. Abelson tyrosine kinase includes sub-types derivatives such as the chimeric fusion (oncoprotein) BCR-Abl with deregulated tyrosine kinase activity or the v-Abl. BCR-Abl is critical in the pathogenesis of 95% of chronic myelogenous leukemia (CML) and 10% of acute lymphocytic leukemia. STI-571 (Gleevec) is an inhibitor of the oncogenic BCR-Abl tyrosine kinase and is used for the treatment of chronic myeloid leukemia (CML). However, some patients in the blast crisis stage of CML are resistant to STI-571 due to mutations in the BCR-Abl kinase. Over 22 mutations have been reported to date with the most common being G250E, E255V, T3151, F317L and M351T.

Compounds of Formula (I), (II), or (III) can inhibit abl kinase, especially v-abl kinase. Compounds of Formula (I), (II), or (III) can also inhibit wild-type BCR-Abl kinase and mutations of BCR-Abl kinase and are thus suitable for the treatment of Bcr-abl-positive cancer and tumor diseases, such as leukemias (especially chronic myeloid leukemia and acute lymphoblastic leukemia, where especially apoptotic mechanisms of action are found), and also shows effects on the subgroup of leukemic stem cells as well as potential for the purification of these cells in vitro after removal of said cells (for example, bone marrow removal) and reimplantation of the cells once they have been cleared of cancer cells (for example, reimplantation of purified bone marrow cells).

PDGF (Platelet-derived Growth Factor) is a very commonly occurring growth factor, which plays an important role both in normal growth and also in pathological cell proliferation, such as is seen in carcinogenesis and in diseases of the smooth-muscle cells of blood vessels, for example in atherosclerosis and thrombosis. Compounds of Formula (I), (II), or (III) can inhibit PDGF receptor (PDGFR) activity and are, therefore, suitable for the treatment of tumor diseases, such as gliomas, sarcomas, prostate cancer, colon cancer, breast cancer, and ovary cancer.

Compounds of Formula (I), (II), or (III), can be used not only as a tumor-inhibiting substance, for example in small cell lung cancer, but also as an agent to treat non-malignant proliferative disorders, such as atherosclerosis, thrombosis, psoriasis, scleroderma, fibrosis, as well as for the protection of stem cells after treatment of chemotherapeutic agents, for example to combat the hemotoxic effect of chemotherapeutic agents, such as 5-fluoruracil, and in asthma. Compounds of Formula (I), (II), or (III) can especially be used for the treatment of diseases, which respond to an inhibition of the PDGF receptor kinase.

Compounds of Formula (I), (II), or (III) can show useful effects in the treatment of disorders arising as a result of transplantation, for example, allogenic transplantation, especially tissue rejection, such as especially obliterative bronchiolitis (OB), i.e. a chronic rejection of allogenic lung transplants. In contrast to patients without OB, those with OB often show an elevated PDGF concentration in bronchoalveolar lavage fluids.

Compounds of Formula (I), (II), or (III) can also be effective in diseases associated with vascular smooth-muscle cell migration and proliferation (where PDGF and PDGF-R often also play a role), such as restenosis and atherosclerosis. These effects and the consequences thereof for the proliferation or migration of vascular smooth-muscle cells in vitro and in vivo can be demonstrated by administration of the compounds of Formula (I), (II), or (III), and also by investigating their effects on the thickening of the vascular intima following mechanical injury in vivo.

Compounds of Formula (I), (II), or (III) can also inhibit cellular processes involving stem-cell factor (SCF, also known as the c-kit ligand or steel factor), such as inhibiting SCF receptor (kit) autophosphorylation and SCF-stimulated activation of MAPK kinase (mitogen-activated protein kinase). MO7e cells are a human promegakaryocytic leukemia cell line, which depends on SCF for proliferation. Compounds of Formula (I), (II), or (III) can inhibit the autophosphorylation of SCF receptors.

The trk family of neurotrophin receptors (trkA, trkB, trkC) promotes the survival, growth and differentiation of the neuronal and non-neuronal tissues. The TrkB protein is expressed in neuroendocrine-type cells in the small intestine and colon, in the alpha cells of the pancreas, in the monocytes and macrophages of the lymph nodes and of the spleen, and in the granular layers of the epidermis (Shibayama and Koizumi, Am J Pathol. 1996 June; 148(6):1807-18). Expression of the TrkB protein has been associated with an unfavorable progression of Wilms tumors and of neuroblastomas. TkrB is, moreover, expressed in cancerous prostate cells but not in normal cells. The signaling pathway downstream of the trk receptors involves the cascade of MAPK activation through the Shc, activated Ras, ERK-1 and ERK-2 genes, and the PLC-gammal transduction pathway (Sugimoto et al., Jpn J Cancer Res. 2001 February; 92(2): 152-60).

The kinase, c-Src transmits oncogenic signals of many receptors. For example, over-expression of EGFR or HER2/neu in tumors leads to the constitutive activation of c-src, which is characteristic for the malignant cell but absent from the normal cell. On the other hand, mice deficient in the expression of c-src exhibit an osteopetrotic phenotype, indicating a key participation of c-src in osteoclast function and a possible involvement in related disorders.

The Tec family kinase, Bmx, a non-receptor protein-tyrosine kinase, controls the proliferation of mammary epithelial cancer cells.

Fibroblast growth factor receptor 3 is shown to exert a negative regulatory effect on bone growth and an inhibition of chondrocyte proliferation. Thanatophoric dysplasia is caused by different mutations in fibroblast growth factor receptor 3, and one mutation, TDII FGFR3, has a constitutive tyrosine kinase activity which activates the transcription factor Stat1, leading to expression of a cell-cycle inhibitor, growth arrest and abnormal bone development (Su et al., Nature, 1997, 386, 288-292). FGFR3 is also often expressed in multiple myeloma-type cancers.

The activity of serum and glucocorticoid-regulated kinase (SGK), is correlated to perturbed ion-channel activities, in particular, those of sodium and/or potassium channels and compounds of Formula (I), (II), or (III) can be useful for treating hypertension.

Lin et al (1997) J. Clin. Invest. 100, 8: 2072-2078 and P. Lin (1998) PNAS 95, 8829-8834, have shown an inhibition of tumor growth and vascularization and also a decrease in lung metastases during adenoviral infections or during injections of the extracellular domain of Tie-2 (Tek) in breast tumor and melanoma xenograft models. Tie2 inhibitors can be used in situations where neovascularization takes place inappropriately (i.e. in diabetic retinopathy, chronic inflammation, psoriasis, Kaposi's sarcoma, chronic neovascularization due to macular degeneration, rheumatoid arthritis, infantile haemangioma and cancers).

Lck plays a role in T-cell signaling. Mice that lack the Lck gene have a poor ability to develop thymocytes. The function of Lck as a positive activator of T-cell signaling suggests that Lck inhibitors may be useful for treating autoimmune diseases such as rheumatoid arthritis.

Multiple forms of p38 MAPK (α, β, γ, δ), each encoded by a separate gene, form part of a kinase cascade involved in the response of cells to a variety of stimuli, including osmotic stress, UV light and cytokine mediated events. These four isoforms of p38 are thought to regulate different aspects of intracellular signaling. Its activation is part of a cascade of signaling events that lead to the synthesis and production of pro-inflammatory cytokines like TNFα. P38 functions by phosphorylating downstream substrates that include other kinases and transcription factors. Agents that inhibitp38 kinase have been shown to block the production of cytokines including but not limited to TNFα, IL-6, IL-8 and IL-1β. Peripheral blood monocytes (PBMCs) have been shown to express and secrete pro-inflammatory cytokines when stimulated with lipopolysaccharide (LPS) in vitro. P38 inhibitors efficiently block this effect when PBMCs are pretreated with such compounds prior to stimulation with LPS. P38 inhibitors are efficacious in animal models of inflammatory disease. The destructive effects of many disease states are caused by the over production of pro-inflammatory cytokines. The ability of p38 inhibitors to regulate this overproduction makes them useful as disease modifying agents.

Molecules that block p38's function have been shown to be effective in inhibiting bone resorption, inflammation, and other immune and inflammation-based pathologies. Thus, a safe and effective p38 inhibitor can provide a means to treat debilitating diseases that can be regulated by modulation of p38 signaling like, for example, RA. Therefore, compounds of Formula (I), (II), or (III) which can inhibit p38 activity are useful for the treatment of inflammation, osteoarthritis, rheumatoid arthritis, cancer, autoimmune diseases, and for the treatment of other cytokine mediated diseases.

JNKs, along with other MAPKs, have been implicated in having a role in mediating cellular response to cancer, thrombin-induced platelet aggregation, immunodeficiency disorders, autoimmune diseases, cell death, allergies, osteoporosis and heart diseases. The therapeutic targets related to activation of the JNK pathway include chronic myelogenous leukemia (CML), rheumatoid arthritis, asthma, osteoarthritis, ischemia, cancer and neurodegenerative diseases. As a result of the importance of JNK activation associated with liver disease or episodes of hepatic ischemia, compounds of Formula (I), (II), or (III) can also be useful to treat various hepatic disorders. A role for JNK in cardiovascular disease such as myocardial infarction or congestive heart failure has also been reported as it has been shown JNK mediates hypertrophic responses to various forms of cardiac stress. It has been demonstrated that the JNK cascade also plays a role in T-cell activation, including activation of the IL-2 promoter. Thus, inhibitors of JNK may have therapeutic value in altering pathologic immune responses. A role for JNK activation in various cancers has also been established, suggesting the potential use of JNK inhibitors in cancer. For example, constitutively activated JNK is associated with HTLV-1 mediated tumorigenesis [Oncogene 13:135-42 (1996)]. JNK may play a role in Kaposi's sarcoma (KS). Other proliferative effects of other cytokines implicated in KS proliferation, such as vascular endothelial growth factor (VEGF), IL-6 and TNF□, may also be mediated by JNK. In addition, regulation of the c-jun gene in p210 BCR-ABL transformed cells corresponds with activity of JNK, suggesting a role for JNK inhibitors in the treatment for chronic myelogenous leukemia (CML) [Blood 92:2450-60 (1998)].

Certain abnormal proliferative conditions are believed to be associated with raf expression and are, therefore, believed to be responsive to inhibition of raf expression. Abnormally high levels of expression of the raf protein are also implicated in transformation and abnormal cell proliferation. These abnormal proliferative conditions are also believed to be responsive to inhibition of raf expression. For example, expression of the c-raf protein is believed to play a role in abnormal cell proliferation since it has been reported that 60% of all lung carcinoma cell lines express unusually high levels of c-raf mRNA and protein. Further examples of abnormal proliferative conditions are hyper-proliferative disorders such as cancers, tumors, hyperplasia, pulmonary fibrosis, angiogenesis, psoriasis, atherosclerosis and smooth muscle cell proliferation in the blood vessels, such as stenosis or restenosis following angioplasty. The cellular signaling pathway of which raf is a part has also been implicated in inflammatory disorders characterized by T-cell proliferation (T-cell activation and growth), such as tissue graft rejection, endotoxin shock, and glomerular nephritis, for example.

The Ras-Raf-MEK-ERK signaling pathway mediates cellular response to growth signals. Ras is mutated to an oncogenic formin-15% of human cancer. The Raf family belongs to the serine/threonine protein kinase and it includes three members, A-Raf, B-Raf and c-Raf (or Raf-1). The focus on Raf being a drug target has centered on the relationship of Raf as a downstream effector of Ras. However, recent data suggests that B-Raf may have a prominent role in the formation of certain tumors with no requirement for an activated Ras allele (Nature 417, 949-954 (1 Jul. 2002). In particular, B-Raf mutations have been detected in a large percentage of malignant melanomas.

Existing medical treatments for melanoma are limited in their effectiveness, especially for late stage melanomas. Compounds of Formula (I), (II), or (III) can also inhibit cellular processes involving b-Raf kinase, providing a new therapeutic opportunity for treatment of human cancers, especially for melanoma.

The stress activated protein kinases (SAPKs) are a family of protein kinases that represent the penultimate step in signal transduction pathways that result in activation of the c-jun transcription factor and expression of genes regulated by c-jun. In particular, c-jun is involved in the transcription of genes that encode proteins involved in the repair of DNA that is damaged due to genotoxic insults. Therefore, agents that inhibit SAPK activity in a cell prevent DNA repair and sensitize the cell to agents that induce DNA damage or inhibit DNA synthesis and induce apoptosis of a cell or that inhibit cell proliferation.

Mitogen-activated protein kinases (MAPKs) are members of conserved signal transduction pathways that activate transcription factors, translation factors and other target molecules in response to a variety of extracellular signals. MAPKs are activated by phosphorylation at a dual phosphorylation motif having the sequence Thr-X-Tyr by mitogen-activated protein kinase kinases (MKKs). In higher eukaryotes, the physiological role of MAPK signaling has been correlated with cellular events such as proliferation, oncogenesis, development and differentiation. Accordingly, the ability to regulate signal transduction via these pathways (particularly via MKK4 and MKK6) could lead to the development of treatments and preventive therapies for human diseases associated with MAPK signaling, such as inflammatory diseases, autoimmune diseases and cancer.

Syk is a tyrosine kinase that plays a critical role in mast cell degranulation and eosinophil activation. Accordingly, Syk kinase is implicated in various allergic disorders, in particular asthma. It has been shown that Syk binds to the phosphorylated gamma chain of the FcεR₁ receptor via N-terminal SH2 domains and is essential for downstream signaling.

Inhibition of eosinophil apoptosis has been proposed as a key mechanism for the development of blood and tissue eosinophilia in asthma. IL-5 and GM-CSF are upregulated in asthma and are proposed to cause blood and tissue eosinophilia by inhibition of eosinophil apoptosis. Inhibition of eosinophil apoptosis has been proposed as a key mechanism for the development of blood and tissue eosinophilia in asthma. It has been reported that Syk kinase is required for the prevention of eosinophil apoptosis by cytokines (Yousefi, et al., J. Exp. Med. 1996; 183: 1407).

The family of human ribosomal S6 protein kinases consists of at least 8 members (RSK1, RSK2, RSK3, RSK4, MSK1, MSK2, p70S6K and p70S6 Kb). Ribosomal protein S6 protein kinases play important pleotropic functions, among them is a key role in the regulation of mRNA translation during protein biosynthesis (Eur. J. Biochem 2000 November; 267(21): 6321-30, Exp Cell Res. Nov. 25, 1999; 253 (1):100-9, Mol Cell Endocrinol. May 25, 1999; 151(1-2):65-77). The phosphorylation of the S6 ribosomal protein by p70S6 has also been implicated in the regulation of cell motility (Immunol. Cell Biol. 2000 August; 78(4):447-51) and cell growth (Prog. Nucleic Acid Res. Mol. Biol., 2000; 65:101-27), and hence, can be important in tumor metastasis, the immune response and tissue repair as well as other disease conditions.

Fes is strongly expressed in myeloid hematopoietic cells and is implicated in both differentiation and survival signaling pathways in myeloid leukocytes. CSK is implicated in cancers, particularly colorectal and breast cancers.

Transforming growth factor-beta (TGFβ) denotes a superfamily of proteins that includes, for example, TGFβ1, TGFβ2, and TGFβ3, which are pleotropic modulators of cell growth and differentiation, embryonic and bone development, extracellular matrix formation, hematopoiesis, immune and inflammatory responses. The members of the TGF family initiate intracellular signaling pathways leading ultimately to the expression of genes that regulate the cell cycle, control proliferative responses, or relate to extracellular matrix proteins that mediate outside-in cell signaling, cell adhesion, migration and intercellular communication. Consequently, compounds of Formula (I), (II), or (III) which can inhibit the TGF intracellular signaling pathway are useful treatments for fibroproliferative diseases, including kidney disorders associated with unregulated TGF activity and excessive fibrosis including glomerulonephritis (GN), such as mesangial proliferative GN, immune GN, and crescentic GN. Other renal conditions include diabetic nephropathy, renal interstitial fibrosis, renal fibrosis in transplant patients receiving cyclosporin, and HIV-associated nephropathy. Collagen vascular disorders include progressive systemic sclerosis, polymyositis, scleroderma, dermatomyositis, eosinophilic fascitis, morphea, or those associated with the occurrence of Raynaud's syndrome. Lung fibroses resulting from excessive TGF activity include adult respiratory distress syndrome, COPD, idiopathic pulmonary fibrosis, and interstitial pulmonary fibrosis often associated with autoimmune disorders, such as systemic lupus erythematosus and scleroderma, chemical contact, or allergies. Another autoimmune disorder associated with fibroproliferative characteristics is rheumatoid arthritis. Fibroproliferative conditions can be associated with surgical eye procedures. Such procedures include retinal reattachment surgery accompanying proliferative vitreoretinopathy, cataract extraction with intraocular lens implantation, and post glaucoma drainage surgery.

In accordance with the foregoing, described are methods for preventing or treating any of the diseases or disorders described above in a subject in need of such treatment, which method comprises administering to said subject a therapeutically effective amount of at least one compound of Formula (I), (II), or (III), or their respective pharmaceutically acceptable derivative thereof. For any of the above uses, the required dosage will vary depending on the mode of administration, the particular condition to be treated and the effect desired.

### Processes for Making Compounds of Formula (I), (II), or (III)

Compounds of Formula (I), (II), and (III) can be synthesized using standard synthetic techniques known to those of skill in the art or using methods known in the art in combination with methods described herein. In additions, solvents, temperatures and other reaction conditions presented herein may vary according to those of skill in the art.

The starting material used for the synthesis of the compounds of Formula (I), (II), and (III) can be obtained from commercial sources, such as Aldrich Chemical Co. (Milwaukee, Wis.), Sigma Chemical Co. (St. Louis, Mo.), or the starting materials can be synthesized. The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference in their entirety). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein. As a guide the following synthetic methods may be utilized.

Formation of Covalent Linkages by Reaction of an Electrophile with a Nucleophile

The compounds described herein can be modified using various electrophiles or nucleophiles to form new functional groups or substituents. Table 1 entitled “Examples of Covalent Linkages and Precursors Thereof” lists selected examples of covalent linkages and precursor functional groups which yield and can be used as guidance toward the variety of electrophiles and nucleophiles combinations available. Precursor functional groups are shown as electrophilic groups and nucleophilic groups.

TABLE 1
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

Use of Protecting Groups

In the reactions described, it may be necessary to protect reactive functional groups, for example hydroxy, amino, imino, thio or carboxy groups, where these are desired in the final product, to avoid their unwanted participation in the reactions. Protecting groups are used to block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd₀-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups may be selected from:

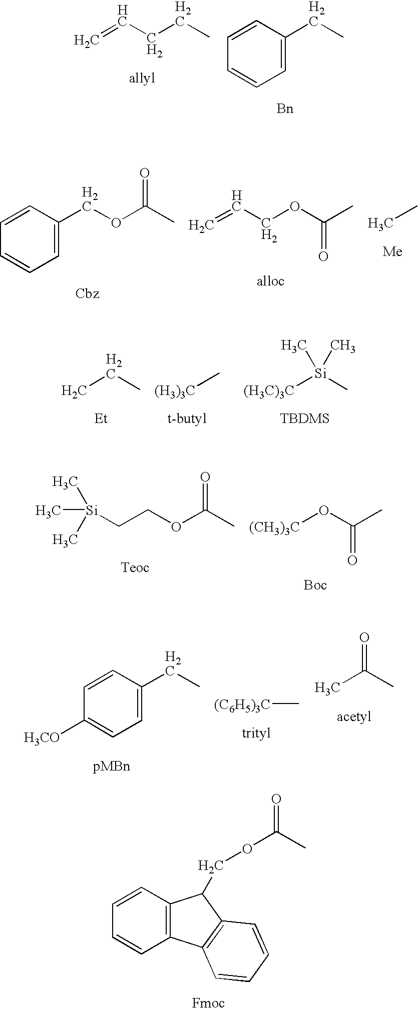

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference in their entirety.

Reaction schemes and representative compounds of Formula (I), (II), or (III) are illustrated in the Examples. In addition, methods of synthesis for various protein kinase inhibitors are described in WO 2005/011597 and WO 2005/034869, which are incorporated by reference in their entirety.

### Further Forms of Compounds

Compounds of Formula (I), (II), or (III) can be prepared as pharmaceutically acceptable salts when an acidic proton present in the parent compound either is replaced by a metal ion, for example an alkali metal ion, an alkaline earth ion, or an aluminum ion; or coordinates with an organic base. In addition, the salt forms of the disclosed compounds can be prepared using salts of the starting materials or intermediates.

Compounds of Formula (I), (II), or (III) can be prepared as a pharmaceutically acceptable acid addition salt (which is a type of a pharmaceutically acceptable salt) by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

Alternatively, compounds of Formula (I), (II), (III) can be prepared as a pharmaceutically acceptable base addition salts (which is a type of a pharmaceutically acceptable salt) by reacting the free acid form of the compound with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds of Formula (I), (II), or (III) can be conveniently prepared or formed during the processes described herein. By way of example only, hydrates of compounds of Formula (I), (II), or (III) can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

Compounds of Formula (I), (II), or (III) include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

Compounds of Formula (I), (II), or (III) in unoxidized form can be prepared from N-oxides of compounds of Formula (I), (II), or (III) by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at 0 to 80° C.

Compounds of Formula (I), (II), or (III) can be prepared as prodrugs. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of Formula (I), (II), or (III) which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Additionally, prodrug derivatives of compounds of Formula (I), (II), or (III) can be prepared by methods known to those of ordinary skill in the art (e.g., for further details see Saulnier et al., (1994), Bioorganic and Medicinal Chemistry Letters, Vol. 4, p. 1985). By way of example only, appropriate prodrugs can be prepared by reacting a non-derivatized compound of Formula (I), (II), or (III) with a suitable carbamylating agent, such as, but not limited to, 1,1-acyloxyalkylcarbanochloridate, para-nitrophenyl carbonate, or the like. Prodrug forms of the herein described compounds, wherein the prodrug is metabolized in vivo to produce a derivative as set forth herein are included within the scope of the claims. Indeed, some of the herein-described compounds may be a prodrug for another derivative or active compound.

Sites on the aromatic ring portion of compounds of Formula (I), (II), or (III) can be susceptible to various metabolic reactions, therefore incorporation of appropriate substituents on the aromatic ring structures, such as, by way of example only, halogens can reduce, minimize or eliminate this metabolic pathway.

The compounds described herein may be labeled isotopically (e.g. with a radioisotope) or by another other means, including, but not limited to, the use of chromophores or fluorescent moieties, bioluminescent labels, or chemiluminescent labels. The compounds of Formula (I), (II), or (III) may possess one or more chiral centers and each center may exist in the R or S configuration. The compounds presented herein include all diastereomeric, enantiomeric, and epimeric forms as well as the appropriate mixtures thereof. Compounds of Formula (I), (II), or (III) can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds described herein, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chiral chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981, herein incorporated by reference in its entirety.

Additionally, the compounds and methods provided herein may exist as geometric isomers. The compounds and methods provided herein include all cis, trans, syn, anti, entgegen (E), and zusammen (Z) isomers as well as the appropriate mixtures thereof. In some situations, compounds may exist as tautomers. All tautomers are included within the formulas described herein are provided by compounds and methods herein. In additional embodiments of the compounds and methods provided herein, mixtures of enantiomers and/or diastereoisomers, resulting from a single preparative step, combination, or interconversion may also be useful for the applications described herein.

### Pharmaceutical Composition/Formulation/Administration

A pharmaceutical composition, as used herein, refers to a mixture of a compound of Formula (I), (II), or (III) with other chemical components, such as carriers, stabilizers, diluents, dispersing agents, suspending agents, thickening agents, and/or excipients. The pharmaceutical composition facilitates administration of the compound to an organism. Pharmaceutical composition containing compounds of Formula (I), (II), or (III) can be administered in therapeutically effective amounts as pharmaceutical compositions by any conventional form and route known in the art including, but not limited to: intravenous, oral, rectal, aerosol, parenteral, ophthalmic, pulmonary, transdermal, vaginal, otic, nasal, and topical administration.

In general, compounds of Formula (I), (II), or (III) will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In some embodiments, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from about 1 to 50 mg active ingredient.

Compounds of Formula (I), (II), or (III) can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising at least one compound of Formula (I), (II), or (III) in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

One may administer the compound in a local rather than systemic manner, for example, via injection of the compound directly into an organ, often in a depot or sustained release formulation. Furthermore, one may administer pharmaceutical composition containing compounds of Formula (I), (II), or (III) in a targeted drug delivery system, for example, in a liposome coated with organ-specific antibody. The liposomes will be targeted to and taken up selectively by the organ. In addition, the pharmaceutical composition containing compounds of Formula (I), (II), or (III) may be provided in the form of a rapid release formulation, in the form of an extended release formulation, or in the form of an intermediate release formulation.

For oral administration, compounds of Formula (I), (II), or (III) can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner. Parental injections may involve for bolus injection or continuous infusion. The pharmaceutical composition of Formula (I), (II), or (III) may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds of Formula (I), (II), or (III) can be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

Suitable formulations for transdermal applications include an effective amount of at least one compound of Formula (I), (II), or (III) with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Formulations suitable for transdermal administration of compounds having the structure of Formula (I), (II), or (III) may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the compounds of Formula (I), (II), or (III) can be accomplished by means of iontophoretic patches and the like. Additionally, transdermal patches can provide controlled delivery of the compounds Formula (I), (II), or (III). The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation, the compounds of Formula (I), (II), or (III) may be in a form as an aerosol, a mist or a powder. Pharmaceutical compositions of Formula (I), (II), or (III) are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

The compounds of Formula (I), (II), or (III) may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In practicing the methods of treatment or use provided herein, therapeutically effective amounts of compounds of Formula (I), (II), or (III) provided herein are administered in a pharmaceutical composition to a mammal having a disease or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions comprising a compound of Formula (I), (II), or (III) may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one pharmaceutically acceptable carrier, diluent or excipient and at least one compound of Formula (I), (II), or (III) described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds may exist as tautomers. All tautomers are included within the scope of the compounds presented herein. Additionally, the compounds described herein can exist in unsolvated as well as solvated forms with pharmaceutically acceptable solvents such as water, ethanol, and the like. The solvated forms of the compounds presented herein are also considered to be disclosed herein. In addition, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

### Methods of Administration and Treatment Methods

Compounds of Formula (I), (II), or (III), and/or their respective pharmaceutically acceptable derivatives thereof, are useful in the treatment or control of cell proliferative disorders, in particular oncological disorders. These compounds and formulations containing said compounds are particularly useful in the treatment or control of solid tumors, such as, for example, breast, colon, lung and prostate tumors. Thus, also described are methods for treating such solid tumors by administering to a patient in need of such therapy an effective amount of a compound of Formula (I), (II), or (III), and/or their respective pharmaceutically acceptable derivatives thereof. Determination of a therapeutically effective amount is within the skill in the art.

The compounds of Formula (I), (II), or (III) can be used in the preparation of medicaments for the treatment of diseases or conditions in which kinase activity contributes to the pathology and/or symptomology of the disease. In addition, a method for treating any of the diseases or conditions described herein in a subject in need of such treatment, involves administration of pharmaceutical compositions containing at least one compound of Formula (I), (II), or (III), or a pharmaceutically acceptable salt, pharmaceutically acceptable N-oxide, pharmaceutically active metabolite, pharmaceutically acceptable prodrug, pharmaceutically acceptable solvate, or other pharmaceutically acceptable derivatives thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

Compositions containing the compound(s) described herein can be used to treat a disease-state or condition including, but not limited to, chronic myeloid leukemia (CML), acute lymphocytic leukemia, reimplantation of purified bone marrow cells, atherosclerosis, thrombosis, gliomas, sarcomas, prostate cancer, colon cancer, breast cancer, and ovary cancer, small cell lung cancer, psoriasis, scleroderma, fibrosis, protection of stem cells after treatment of chemotherapeutic agents, asthma, allogenic transplantation, tissue rejection, obliterative bronchiolitis (OB), restenosis, Wilms tumors, neuroblastomas, mammary epithelial cancer cells, thanatophoric dysplasia, growth arrest, abnormal bone development, myeloma-type cancers, hypertension, diabetic retinopathy, psoriasis, Kaposi's sarcoma, chronic neovascularization due to macular degeneration, rheumatoid arthritis, infantile haemangioma, rheumatoid arthritis, other autoimmune diseases, thrombin-induced platelet aggregation, immunodeficiency disorders, allergies, osteoporosis, osteoarthritis, neurodegenerative diseases, hepatic ischemia, myocardial infarction, congestive heart failure, other heart diseases, HTLV-1 mediated tumorigenesis, hyperplasia, pulmonary fibrosis, angiogenesis, stenosis, endotoxin shock, glomerular nephritis, genotoxic insults, chronic inflammation, and other inflammatory diseases, in a patient in need of such treatment, the method comprising administering to the patient an effective amount of a compound described herein, or a tautomer, prodrug, solvate, or salt thereof.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition. In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disease or condition is retained. Patients can, however, require intermittent treatment on a long-term basis upon any recurrence of symptoms.

In certain instances, it may be appropriate to administer therapeutically effective amounts of at least one of the compounds described herein (or a pharmaceutically acceptable salts, pharmaceutically acceptable N-oxides, pharmaceutically active metabolites, pharmaceutically acceptable prodrugs, pharmaceutically acceptable solvates, and other pharmaceutically acceptable derivates thereof) in combination with another therapeutic agent. By way of example only, if one of the side effects experienced by a patient upon receiving one of the compounds herein is inflammation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit.

In any case, regardless of the disease or condition being treated, the overall benefit experienced by the patient may simply be additive of the two therapeutic agents or the patient may experience a synergistic benefit. For example, synergistic effects can occur with other immunomodulatory or anti-inflammatory substances, for example when used in combination with cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Where compounds of Formula (I), (II), or (III) are administered in conjunction with other therapies, dosages of the co-administered compounds will of course vary depending on the type of co-drug employed, on the specific drug employed, on the condition being treated and so forth.

For example, synergistic effects can also occur with compounds of Formula (I), (II), or (III) and other substances used in the treatment of hypokalemia, hypertension, congestive heart failure, renal failure, in particular chronic renal failure, restenosis, atherosclerosis, syndrome X, obesity, nephropathy, post-myocardial infarction, coronary heart disease, increased formation of collagen, fibrosis and remodeling following hypertension and endothelial dysfunction. Examples of such compounds include anti-obesity agents, such as orlistat, anti-hypertensive agents, inotropic agents and hypolipidemic agents including, but not limited to, loop diuretics, such as ethacrynic acid, furosemide and torsemide; angiotensin converting enzyme (ACE) inhibitors, such as benazepril, captopril, enalapril, fosinopril, lisinopril, moexipril, perinodopril, quinapril, ramipril and trandolepril; inhibitors of the Na-K-ATPase membrane pump, such as digoxin; neutralendopeptidase (NEP) inhibitors; ACE/NEP inhibitors, such as omapatrilat, sampatrilat, and fasidotril; angiotensin II antagonists, such as candesartan, eprosartan, irbesartan, losartan, telmisartan and valsartan, in particularvalsartan; □-adrenergic receptor blockers, such as acebutolol, betaxolol, bisoprolol, metoprolol, nadolol, propanolol, sotalol and timolol; inotropic agents, such as digoxin, dobutamine and milrinone; calcium channel blockers, such as amlodipine, bepridil, diltiazem, felodipine, nicardipine, nimodipine, nifedipine, nisoldipine and verapamil; and 3-hydroxy-3-methyl-glutaryl coenzyme A reductase (HMG-CoA) inhibitors, such as lovastatin, pitavastatin, simvastatin, pravastatin, cerivastatin, mevastatin, velostatin, fluvastatin, dalvastatin, atorvastatin, rosuvastatin and rivastatin. Where the compounds described herein are administered in conjunction with other therapies, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is one of the compounds described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; we envision the use of multiple therapeutic combinations.

In addition, the compounds of Formula (I), (II), or (III) may also be used in combination with procedures that may provide additional or synergistic benefit to the patient. By way of example only, patients are expected to find therapeutic and/or prophylactic benefit in the methods described herein, wherein pharmaceutical compositions containing compounds of Formula (I), (II), or (III) and/or combinations with other therapeutics are combined with genetic testing to determine whether that individual is a carrier of a mutant gene that is known to be correlated with certain diseases or conditions.

The compounds of Formula (I), (II), or (III) and combination therapies can be administered before, during or after the occurrence of a disease or condition, and the timing of administering the composition containing a compound can vary. Thus, for example, the compounds can be used as a prophylactic and can be administered continuously to subjects with a propensity to conditions or diseases in order to prevent the occurrence of the disease or condition. The compounds and compositions can be administered to a subject during or as soon as possible after the onset of the symptoms. The administration of the compounds can be initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. A compound is preferably administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the compound or a formulation containing the compound can be administered for at least 2 weeks, preferably about 1 month to about 5 years, and more preferably from about 1 month to about 3 years.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

In some embodiments, the daily dosages appropriate for the compounds of Formula (I), (II), or (III) described herein are from about 0.03 to 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in retard form. Suitable unit dosage forms for oral administration comprise from about 1 to 50 mg active ingredient. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Kits/Articles of Manufacture

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit will typically may comprise one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

### EXAMPLES

The following examples provide illustrative methods for making and testing the effectiveness and safety of the compounds of Formula (I), (II), or (III). These examples are provided for illustrative purposes only and not to limit the scope of the claims provided herein. All of the methods disclosed and claimed herein can be made and executed without undue experimentation in light of the present disclosure. It will be apparent to those of skill in the art that variations may be applied to the methods and in the steps or in the sequence of steps of the method described herein without departing from the concept, spirit and scope of the claims. All such similar substitutes and modifications apparent to those skilled in the art are deemed to be within the spirit, scope and concept of the appended claims.

### Example 1

### Synthesis of 6-Chloro-4-ethylamino-pyridine-3-carbaldehyde

Chemical structure of 6-Chloro-4-ethylamino-pyridine-3-carbaldehyde is shown below, and Scheme 1 illustrates various steps for preparing intermediate compounds.

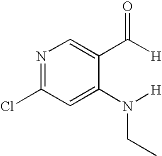

### 6-Chloro-4-ethylamino-pyridine-3-carbaldehyde

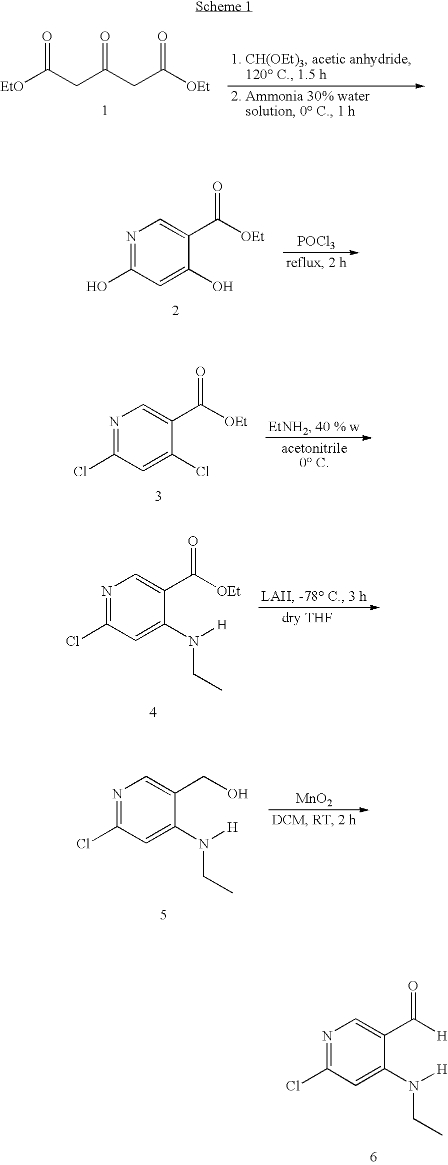

### Example 1a

### Preparation of 4,6-Dihydroxy-nicotinic acid ethyl ester

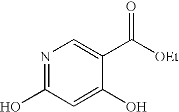

### 4,6-Dihydroxy-nicotinic acid ethyl ester

Mix the diethyl 1,3-acetonedicarboxylate (10.11 g, 50 mmol) with triethyl orthoformate (8.14 g, 55 mmol) and acetic anhydride (10.20 g, 100 mmol) in a 100 ml flask and heat up to 120° C. for 1.5 hours. The crude product is distilled under vacuum (150-200 mmHg) around 90-100° C., the light yellow oil solution is collected in the condenser. The left residue is cooled in ice and mixed with 30% ammonia (4 ml). The reaction is continued in ice bath for 1 hour and then acidified with 2N HCl to pH<5. Remove the solvent under the vacuum. The crude product is purified by flash chromatography using EA/Hexane (1:1). The final product 4,6-dihydroxy-nicotinic acid ethyl ester is a clear oil, 2.85 g.

### Example 1b

### Preparation of 4,6-Dichloro-nicotinic acid ethyl ester

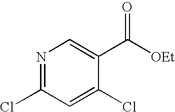

### 4,6-Dichloro-nicotinic acid ethyl ester

4,6-Dihydroxy-nicotinic acid ethyl ester (2.85 g) is mixed with pure POCl₃ 25 ml in a 100 ml flask and heated up to 110° C. for 2 hours. After cooling down, most of the POCl₃ is removed under vacuum. The crude dark color product is pooled into small amount ice-water mixture, and neutralized with saturated sodium carbonate solution. Extract the product by using 200 ml ethyl acetate for a couple of times. The combined organic layer is washed by saturated sodium chloride solution and dried by Na₂SO₄. After removing the solvent, the crude product is purified by flash chromatography using EA/Hexane (15:85). The final product 4,6-dichloro-nicotinic acid ethyl ester is a white solid, 3.05 g.

### Example 1c

### Preparation of 6-Chloro-4-ethylamino-nicotinic acid ethyl ester

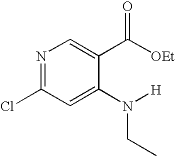

### 6-Chloro-4-ethylamino-nicotinic acid ethyl ester

4,6-Dichloro-nicotinic acid ethyl ester (2.19 g, 10 mmol) is dissolved in 30 ml acetonitrile and cooled down to 0° C., slowly add 4 ml ethylamine solution (40% ethylamine water solution, 50 mmol). The reaction is stirred at 0° C. for 30 minutes and warmed up to RT for another 2 hours. Remove the solvent under the vacuum and purify the crude product by flash chromatography using EA/Hexane (30:70). The final product 6-chloro-4-ethylamino-nicotinic acid ethyl ester is a white solid, 2.03 g.

### Example 1d

### Preparation of (6-Chloro-4-ethylamino-pyridin-3-yl)-methanol

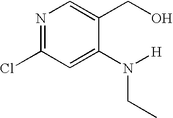

### (6-Chloro-4-ethylamino-pyridin-3-yl)-methanol

6-chloro-4-ethylamino-nicotinic acid ethyl ester (2.03 g, 9.5 mmol) is dissolved in 30 ml anhydrous THF and cooled down to −78° C. Add 20 ml LAH THF solution (1M THF solution, 20 mmol) slowly and continue the reaction for 3 hours at −78° C. Warm up the reaction to the RT slowly and check TLC to make sure no starting materials left. Add small amount MeOH/EA (1:1) mixture slowly to destroy the excess LAH. The crude product goes through a celite plug and is washed by EA for a couple of times. After removing the solvent under vacuum, the crude product is purified by flash chromatography using MeOH/DCM (5%:95%). The final product (6-chloro-4-ethylamino-pyridin-3-yl)-methanol is a white solid, 1.40 g.

### Example 1e

### Preparation of 6-Chloro-4-ethylamino-pyridine-3-carbaldehyde

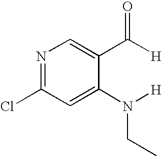

### 6-Chloro-4-ethylamino-pyridine-3-carbaldehyde

(6-chloro-4-ethylamino-pyridin-3-yl)-methanol (1.40 g, 8.1 mol) is dissolved in 40 ml DCM and 7.0 g MnO₂ (81 mmol) is added. The reaction is stirred at RT for 2 hours. Then the reaction solution goes through a celite plug and washed by EA. After removing the solvent under the vacuum, the crude product is purified by flash chromatography using EA/Hexane (3:7). The final product 6-chloro-4-ethylamino-pyridine-3-carbaldehyde is a white solid, 1.30 g.

### Example 2

### Synthesis of 3-Cyanomethyl-5-methoxy-benzoic acid methyl ester

Chemical structure of 3-Cyanomethyl-5-methoxy-benzoic acid methyl ester is shown below, and Scheme 2 illustrates various steps for preparing intermediate compounds.

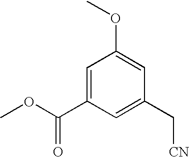

### 3-Cyanomethyl-5-methoxy-benzoic acid methyl ester

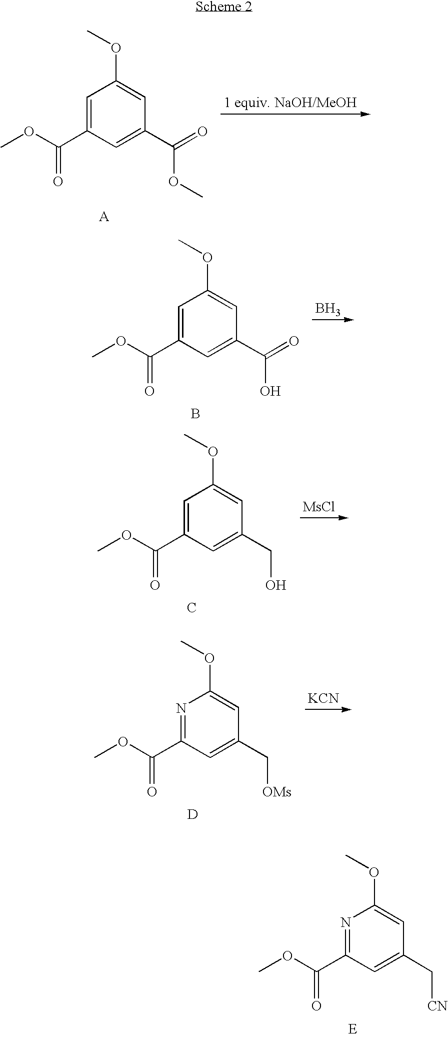

### Example 2a

### Preparation of 5-Methoxy-isophthalic acid monomethyl ester

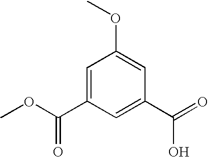

### 5-Methoxy-isophthalic acid monomethyl ester

5-Methoxy-isophthalic acid dimethyl ester (5 g, 22.3 mmol) and NaOH (0.892 g, 22.3 mmol) is mixed in 50 ml methanol and refluxed at 80° C. overnight. The reaction mixture is cooled to room temperature and solvent is removed by rotary evaporation. The solid is treated with HCl and the solid is collected by filtration, washed with water and dried under vacuum to give 5-Methoxy-isophthalic acid monomethyl ester as white solid (4.0 g, 85%).

### Example 2b

### Preparation of 3-Hydroxymethyl-5-methoxy-benzoic acid methyl ester

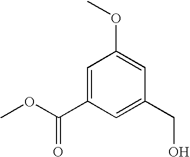

3-Hydroxymethyl-5-methoxy-benzoic acid methyl ester

5-Methoxy-isophthalic acid monomethyl ester (4 g, 19 mmol) is dissolved in 25 ml dry THF and then 25 ml of 1N borane in THF is added dropwise at room temperature. The reaction is stirred at room temperature for 30 min. The solvent is removed by rotary evaporation. The crude product is purified by silica gel flash chromatography to give 3-Hydroxymethyl-5-methoxy-benzoic acid methyl ester (2.9 g, 78%).

### Example 2c

### Preparation of 3-Methanesulfonyloxymethyl-5-methoxy-benzoic acid methyl ester

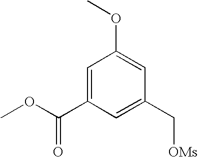

### 3-Methanesulfonyloxymethyl-5-methoxy-benzoic acid methyl ester

3-Hydroxymethyl-5-methoxy-benzoic acid methyl ester (2.9 g, 14.8 mmol) is dissolved in 80 ml dry methylene chloride, cooled to 0° C., followed by addition of 1.2 equivalent of TEA and 1.15 equivalent of MsCl. The reaction is stirred on ice for 30 min followed by room temperature for 2 hours. After the reaction is completed, 80 ml 10% NaHCO₃ solution is added to the reaction mixture. The reaction mixture is extracted three times with 80 ml methylene chloride. The organic phase is combined and washed with brine and dried over Na₂SO₄. The crude product is used without further purification.

### Example 2d

### Preparation of 3-Cyanomethyl-5-methoxy-benzoic acid methyl ester

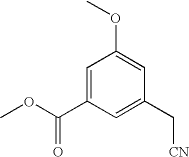

### 3-Cyanomethyl-5-methoxy-benzoic acid methyl ester

3-Methanesulfonyloxymethyl-5-methoxy-benzoic acid methyl ester (4 g, 14 mmol) is dissolved in 50 ml of DMF and 1.4 g KCN is added at 0° C. The reaction is warmed up to room temperature and stirred overnight. After the reaction is complete, 120 ml water is added and the reaction mixture is extracted with 100 ml ether three times. The organic phase is combined and washed with brine, dried with Na₂SO₄. The crude product is purified by silica gel flash chromatography to give final product (2.1 g, 71%); ¹H NMR acetone-d₆, δ7.65 (s, 1H), 7.49 (s, 1H), 7.25 (s, 1H), 4.05 (s, 2H), 3.91 (m, 6H).

### Example 3

### Synthesis of 3-(1-Ethyl-7-ethylamino-2-oxo-1,2-dihydro-[1,6]naphthyridin-3-yl)-5,N-dimethoxy-benzamide

3-(1-Ethyl-7-ethylamino-2-oxo-1,2-dihydro-[1,6]naphthyridin-3-yl)-5,N-dimethoxy-benzamide can be prepared using 6-Chloro-4-ethylamino-pyridine-3-carbaldehyde from Example 1 and 3-Cyanomethyl-5-methoxy-benzoic acid methyl ester from Example 2 as starting materials. Scheme 3 illustrates various steps for preparing intermediate compounds.

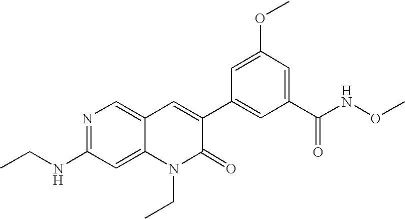

### 3-(1-Ethyl-7-ethylamino-2-oxo-1,2-dihydro-[1,6]naphthyridin-3-yl)-5,N-dimethoxy-benzamide

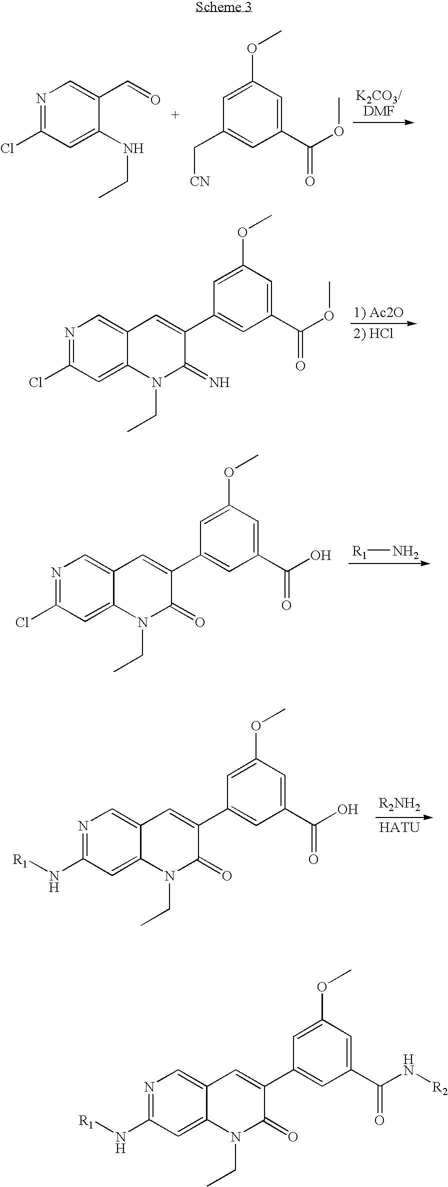

### Example 3a

### Preparation of 3-(7-Chloro-1-ethyl-2-imino-1,2-dihydro-[1,6]naphthyridin-3-yl)-5-methoxy-benzoic acid methyl ester

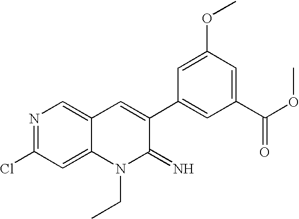

### 3-(7-Chloro-1-ethyl-2-imino-1,2-dihydro-[1,6]naphthyridin-3-yl)-5-methoxy-benzoic acid methyl ester

6-Chloro-4-ethylamino-pyridine-3-carbaldehyde (370 mg, 2 mmol), 3-Cyanomethyl-5-methoxy-benzoic acid methyl ester (410 mg, 2 mmol) and K₂CO₃ (0.9 g, 6 mmol) are mixed in 10 ml dry DMF and stirred at 100° C. for 8 hours. The reaction mixture is diluted into 70 ml water and extracted with 80 ml ethyl acetate three times. The organic phase is combined and washed with brine, dried over Na₂SO₄. The crude product is purified by silica gel flash chromatography, eluted with 40% ethyl acetate in hexane to give 3-(7-Chloro-1-ethyl-2-imino-1,2-dihydro-[1,6]naphthyridin-3-yl)-5-methoxy-benzoic acid methyl ester (550 mg, 74%).

### Example 3b

### Preparation of 3-(7-Chloro-1-ethyl-2-oxo-1,2-dihydro-[1,6]naphthyridin-3-yl)-5-methoxy-benzoic acid

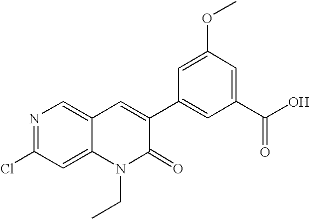

### 3-(7-Chloro-1-ethyl-2-oxo-1,2-dihydro-[1,6]naphthyridin-3-yl)-5-methoxy-benzoic acid

3-(7-Chloro-1-ethyl-2-imino-1,2-dihydro-[1,6]naphthyridin-3-yl)-5-methoxy-benzoic acid methyl ester (500 mg, 1.35 mmol) in 5 ml acetic anhydride is stirred at 120° C. for 2 hours. The acetic anhydride is removed by rotary evaporation. To the flask containing the residue is added 5 ml of 6N HCl. The reaction is stirred at 80° C. for 8 hours. The reaction is cooled down to 0° C. and then certain amount (˜15 ml) of 1N NaOH is added until there is precipitation. The solid is collected by filtration, washed with water and taken to dryness to give 3-(7-Chloro-1-ethyl-2-oxo-1,2-dihydro-[1,6]naphthyridin-3-yl)-5-methoxy-benzoic acid (420 mg, 87%).

### Example 3c

### Preparation of 3-(1-Ethyl-7-ethylamino-2-oxo-1,2-dihydro-[1,6]naphthyridin-3-yl)-5-methoxy-benzoic acid

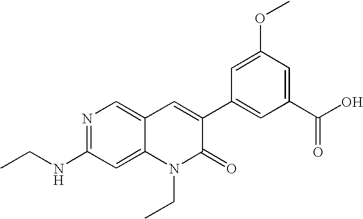

3-(1-Ethyl-7-ethylamino-2-oxo-1,2-dihydro-[1,6]naphthyridin-3-yl)-5-methoxy-benzoic acid

3-(7-Chloro-1-ethyl-2-oxo-1,2-dihydro-[1,6]naphthyridin-3-yl)-5-methoxy-benzoic acid (180 mg, 0.48 mmol), ethylamine (1 ml of 70% aqueous solution) and 1 ml of 2-methoxyethanol are added to a sealed tube. The reaction is stirred at 110° C. for 8 hours. The solvent is removed by rotary evaporation. The residue is treated with 5 ml 0.1N HCl and sonicated briefly. The solid is collected by filtration and washed with water and dried under vacuum to give 3-(1-Ethyl-7-ethylamino-2-oxo-1,2-dihydro-[1,6]naphthyridin-3-yl)-5-methoxy-benzoic acid (140 mg, 76%).

### Example 3d

### Preparation of 3-(1-Ethyl-7-ethylamino-2-oxo-1,2-dihydro-[1,6]naphthyridin-3-yl)-5,N-dimethoxy-benzamide

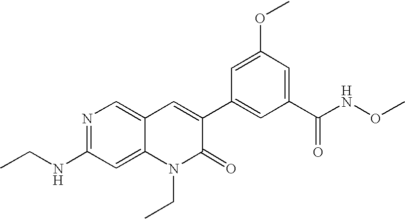

### 3-(1-Ethyl-7-ethylamino-2-oxo-1,2-dihydro-[1,6]naphthyridin-3-yl)-5,N-dimethoxy-benzamide

3-(1-Ethyl-7-ethylamino-2-oxo-1,2-dihydro-[1,6]naphthyridin-3-yl)-5-methoxy-benzoic acid (15 mg, 0.04 mmol), HATU (17 mg, 0.044 mmol), methoxylamine hydrochloride (10 mg, 0.12 mmol) and DIEA (42 μl, 0.24 mmol) are mixed in 0.5 ml DMF. The reaction is stirred at room temperature for 2 hours. The solvent is removed by rotary evaporation. The crude product is purified by RP-HPLC to give 3-(1-Ethyl-7-ethylamino-2-oxo-1,2-dihydro-[1,6]naphthyridin-3-yl)-5,N-dimethoxy-benzamide as light yellow solid (12 mg, 74%); ¹H NMR 400 MHz (DMSO-d₆) δ 11.99 (s, 1H), 8.70 (s, 1H), 8.27 (s, 1H), 7.82 (s, 1H), 7.63 (s, 1H), 7.47 (s, 1H), 6.62 (s, 1H), 4.38 (q, 2H, J=7.2 Hz), 4.03 (s, 3H), 3.92 (s, 3H), 3.58 (q, 2H, J=7.2 Hz), 3.37 (s, 1H), 1.42 (m, 6H); MS m/z 397.2 (M+1).

### Example 4

### Synthesis of N-Ethoxy-3-[8-ethyl-2-(4-morpholin-4-yl-phenylamino)-7-oxo-7,8-dihydro-pyrido[2,3-d]pyrimidin-6-yl]-5-methoxy-benzamide

N-Ethoxy-3-[8-ethyl-2-(4-morpholin-4-yl-phenylamino)-7-oxo-7,8-dihydro-pyrido[2,3-d]pyrimidin-6-yl]-5-methoxy-benzamide can be prepared using 3-Cyanomethyl-5-methoxy-benzoic acid methyl ester from Example 2 and 4-Ethylamino-2-methylsulfanyl-pyrimidine-5-carbaldehyde as starting materials. Scheme 4 illustrates various steps for preparing intermediate compounds.

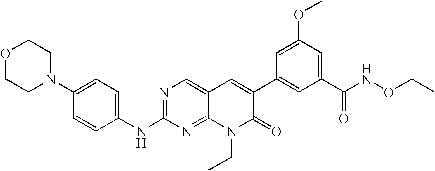

### N-Ethoxy-3-[8-ethyl-2-(4-morpholin-4-yl-phenylamino)-7-oxo-7,8-dihydro-pyrido[2,3-d]pyrimidin-6-yl]-5-methoxy-benzamide

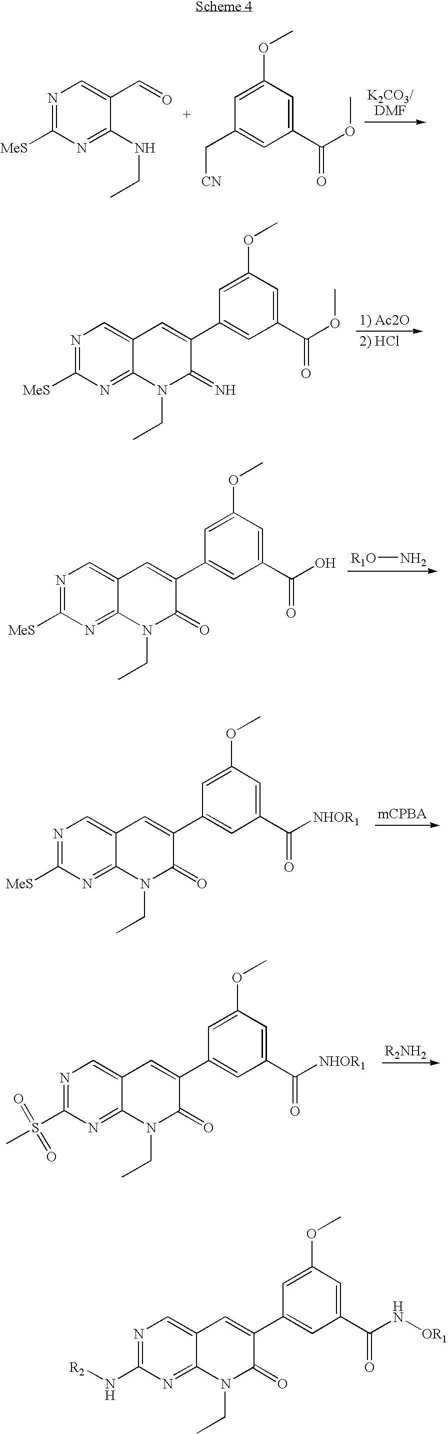

### Example 4a

### Preparation of 3-(8-Ethyl-7-imino-2-methylsulfanyl-7,8-dihydro-pyrido[2,3-d]pyrimidin-6-yl)-5-methoxy-benzoic acid methyl ester

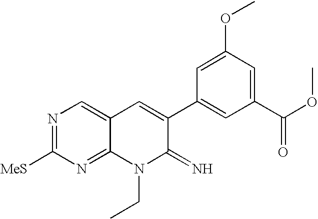

### 3-(8-Ethyl-7-imino-2-methylsulfanyl-7,8-dihydro-pyrido[2,3-d]pyrimidin-6-yl)-5-methoxy-benzoic acid methyl ester

4-Ethylamino-2-methylsulfanyl-pyrimidine-5-carbaldehyde (524 mg, 2.65 mmol), 3-cyanomethyl-5-methoxy-benzoic acid methyl ester (653 mg, 3.18 mmol) and K₂CO₃ (0.917 g, 6.63 mmol) are mixed in 10 ml dry DMF and stirred at 120° C. for 3 hours. The reaction mixture is diluted into 70 ml with water. The solid is collected by filtration, washed with water, dried to give 3-(8-Ethyl-7-imino-2-methylsulfanyl-7,8-dihydro-pyrido[2,3-d]pyrimidin-6-yl)-5-methoxy-benzoic acid methyl ester (706 mg, 70%); MS m/z 385.10 (M+1).

### Example 4b

### Preparation of 3-(8-Ethyl-2-methylsulfanyl-7-oxo-7,8-dihydro-pyrido[2,3-d]pyrimidin-6-yl)-5-methoxy-benzoic acid

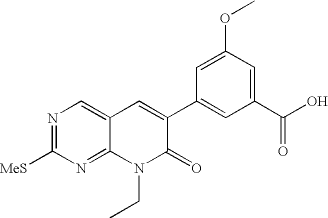

### 3-(8-Ethyl-2-methylsulfanyl-7-oxo-7,8-dihydro-pyrido[2,3-d]pyrimidin-6-yl)-5-methoxy-benzoic acid

3-(8-Ethyl-7-imino-2-methylsulfanyl-7,8-dihydro-pyrido[2,3-d]pyrimidin-6-yl)-5-methoxy-benzoic acid methyl ester (577 mg, 1.50 mmol) in 10 ml acetic anhydride is stirred at 105° C. for 1 hour. The reaction mixture is cooled to room temperature and 10 ml of 6N HCl is added. After stirring at 105° C. for 1 hour, the reaction mixture is cooled down to room temperature and diluted with water. The solid is collected by filtration, washed with water and taken to dryness to give 3-(8-Ethyl-2-methylsulfanyl-7-oxo-7,8-dihydro-pyrido[2,3-d]pyrimidin-6-yl)-5-methoxy-benzoic acid, which is used for next reaction without further purification; MS m/z 372.10 (M+1).

### Example 4c

### Preparation of N-Ethoxy-3-(8-ethyl-2-methylsulfanyl-7-oxo-7,8-dihydro-pyrido[2,3-d]pyrimidin-6-yl)-5-methoxy-benzamide

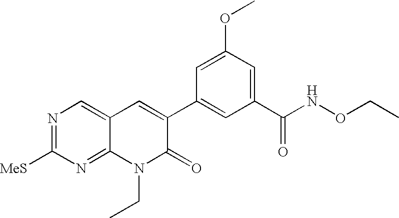

### N-Ethoxy-3-(8-ethyl-2-methylsulfanyl-7-oxo-7,8-dihydro-pyrido[2,3-d]pyrimidin-6-yl)-5-methoxy-benzamide

DIEA is added to a solution of 3-(8-ethyl-2-methylsulfanyl-7-oxo-7,8-dihydro-pyrido[2,3-d]pyrimidin-6-yl)-5-methoxy-benzoic acid (256 mg, 0.69 mmol), HATU (288 mg, 0.757 mmol) in DMF (10 ml) at 0° C. After stirring for 15 minutes, ethoxyl amine hydrochloride (110 mg, 1.13 mmol) are added. The reaction is stirred at room temperature for 1 hour. The solvent is removed by rotary evaporation, saturated Na₂CO₃ solution is added to the residue. The solid is collected by filtration, washed with water and taken to dryness to give N-Ethoxy-3-(8-ethyl-2-methylsulfanyl-7-oxo-7,8-dihydro-pyrido[2,3-d]pyrimidin-6-yl)-5-methoxy-benzamide, 276 mg (97% yield), which is used for next reaction without further purification; MS m/z 415.14 (M+1).

### Example 4d

### Preparation of N-Ethoxy-3-(8-ethyl-2-methanesulfonyl-7-oxo-7,8-dihydro-pyrido[2,3-d]pyrimidin-6-yl)-5-methoxy-benzamide

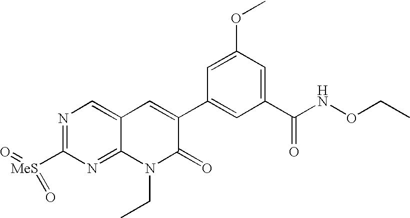

### N-Ethoxy-3-(8-ethyl-2-methanesulfonyl-7-oxo-7,8-dihydro-pyrido[2,3-d]pyrimidin-6-yl)-5-methoxy-benzamide

A solution of N-ethoxy-3-(8-ethyl-2-methanesulfonyl-7-oxo-7,8-dihydro-pyrido[2,3-d]pyrimidin-6-yl)-5-methoxy-benzamide (136.5 mg, 0.33 mmol) in DCM (10 ml) and DMF (0.5 ml) is cooled to 0° C.; mCPBA (190 mg, 0.847 mmol) is added portionwise. The reaction mixture is allowed to warm to room temperature. After stirring overnight, the reaction mixture is diluted with DCM and quenched with 20 ml of 5% Na₂S₂O₃ solution. The organic phase is separated and washed with saturated Na₂CO₃ solution, brine and dried over Na₂SO₄, concentrated to afford N-Ethoxy-3-(8-ethyl-2-methanesulfonyl-7-oxo-7,8-dihydro-pyrido[2,3-d]pyrimidin-6-yl)-5-methoxy-benzamide 123 mg (84%), which is used for next reaction; MS m/z 447.1 (M+1).

### Example 4e

### Preparation of N-Ethoxy-3-[8-ethyl-2-(4-morpholin-4-yl-phenylamino)-7-oxo-7,8-dihydro-pyrido[2,3-d]pyrimidin-6-yl]-5-methoxy-benzamide

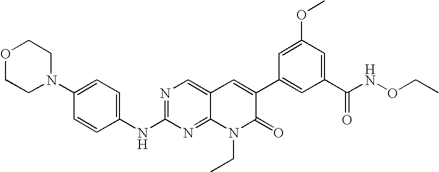

### N-Ethoxy-3-[8-ethyl-2-(4-morpholin-4-yl-phenylamino)-7-oxo-7,8-dihydro-pyrido[2,3-d]pyrimidin-6-yl]-5-methoxy-benzamide

A mixture of N-ethoxy-3-(8-ethyl-2-methanesulfonyl-7-oxo-7,8-dihydro-pyrido[2,3-d]pyrimidin-6-yl)-5-methoxy-benzamide (27 mg, 0.06 mol), morpholin-4-yl-phenylamine (44 mg, 0.24 mol) in 1,3-dimethyl-2-imidazolidinone (0.5 ml) is heated at 100° C. for 24 hours. The crude product is purified by RP-HPLC to give N-Ethoxy-3-[8-ethyl-2-(4-morpholin-4-yl-phenylamino)-7-oxo-7,8-dihydro-pyrido[2,3-d]pyrimidin-6-yl]-5-methoxy-benzamide as free base; ¹H NMR 400 MHz (DMSO-d₆) δ 11.69 (s, 1H), 9.98 (s, 1H), 8.80 (s, 1H), 8.08 (s, 1H), 7.69 (d, 2H, J=8.8 Hz), 7.64 (s, 1H), 7.45 (s, 1H), 7.28 (s, 1H), 6.96 (d, 2H, J=8.8 Hz), 4.40 (q, 2H, J=6.8 Hz), 3.96 (q, 2H, J=6.8 Hz), 3.84 (s, 3H), 3.75 (m, 4H), 3.08 (m, 4H), 1.30 (t, 3H, J=6.8 Hz), 1.24 (t, 3H, J=6.8 Hz); MS m/z 545.2 (M+1).

### Example 5

### Synthesis of 3-(7-Cyclopropylamino-1-ethyl-2-oxo-1,4-dihydro-2H-pyrimido[4,5-d]pyrimidin-3-yl)-N-ethoxy-5-methoxy-benzamide

3-(7-Cyclopropylamino-1-ethyl-2-oxo-1,4-dihydro-2H-pyrimido[4,5-d]pyrimidin-3-yl)-N-ethoxy-5-methoxy-benzamide can be prepared using 5-Hydroxymethyl-1H-pyrimidine-2,4-dione as a starting material. Scheme 5 illustrates various steps for preparing intermediate compounds.

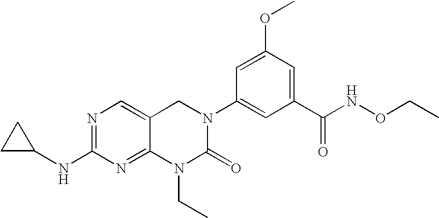

### 3-(7-Cyclopropylamino-1-ethyl-2-oxo-1,4-dihydro-2H-pyrimido[4,5-d]pyrimidin-3-yl)-N-ethoxy-5-methoxy-benzamide

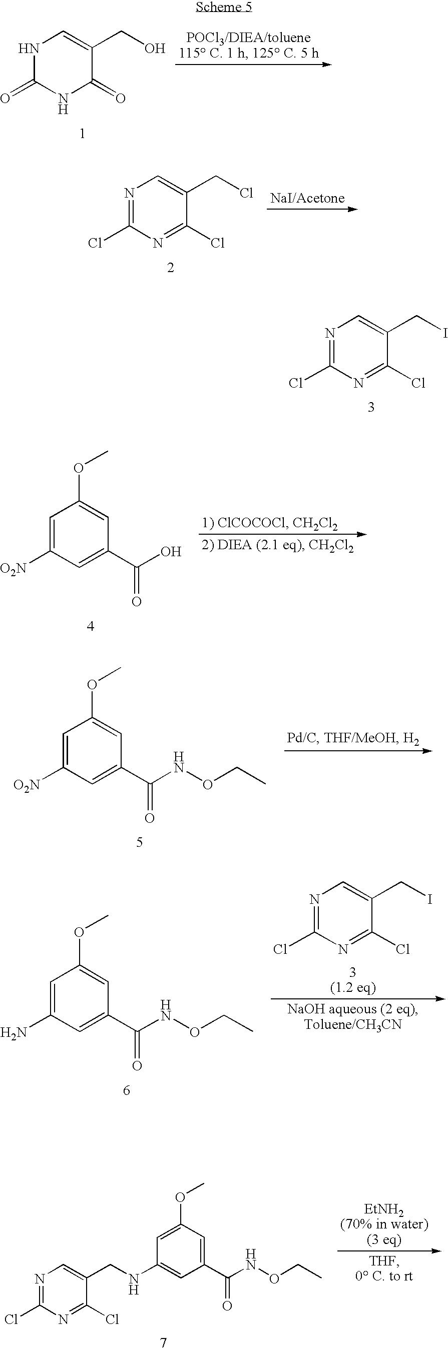

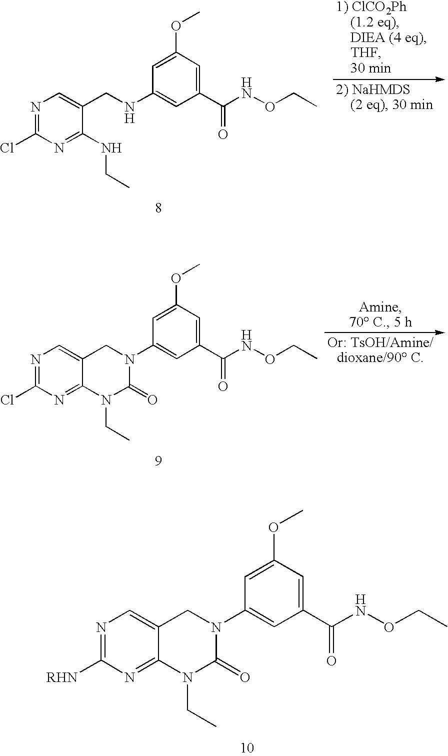

### Example 5a

### Preparation of 2,4-Dichloro-5-chloromethyl-pyrimidine

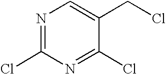

### 2,4-Dichloro-5-chloromethyl-pyrimidine

To a flask containing 5-Hydroxymethyl-1H-pyrimidine-2,4-dione (20 g, 140.7 mmol), phosphorous oxychloride (65.9 ml, 282.7 mmol) and toluene (40 ml) are added. The mixture is cooled with an ice-water bath, then N,N-diisopropylethylamine (73.9 ml, 424.1 mmol) is added slowly over 5 mins. After completion of the addition, the cooling bath is removed and the mixture is heated at 115° C. for 1 hour, then 125° C. for 5 hours. TLC analysis indicated reaction is complete. After the reaction is cooled to room temperature, the mixture is cautiously added into a stirred bi-phasic mixture of water (120 ml) and ethyl acetate (90 ml), using a ice-water bath. After the mixture is stirred for 60 mins with ice-water bath, the mixture is extracted with toluene (4×60 ml). The combined organic layers are dried, filtered, then concentrated to dryness under reduced pressure. Further purification is done using a short silica gel column, affording 2,4-Dichloro-5-chloromethyl-pyrimidine as a white solid (23.06 g, 83%); ¹H NMR 400 MHz (CDCl₃) δ 8.67 (s, 1H), 4.65 (s, 2H).

### Example 5b

### Preparation of 2,4-Dichloro-5-iodomethyl-pyrimidine

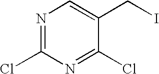

### 2,4-Dichloro-5-iodomethyl-pyrimidine

A mixture of 2,4-Dichloro-5-chloromethyl-pyrimidine (10 g, 50.6 mmol), sodium iodide (7.69 g, 51.3 mmol) in acetone (60 ml) is stirred at room temperature for 20 min, then refluxed for 15 min. The reaction is allowed to cool to room temperature, then the solid is filtered and washed by acetone. The filtrate is concentrated to afford 2,4-Dichloro-5-iodomethyl-pyrimidine as pale yellow solid (14.6 g, 100%); ¹H NMR 400 MHz (CDCl₃) δ 8.54 (s, 1H), 4.33 (s, 2H); MS m/z 288.9 (M+1).

### Example 5c

### Preparation of N-Ethoxy-3-methoxy-5-nitro-benzamide

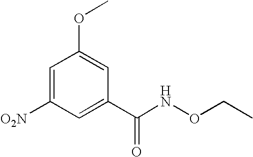

### N-Ethoxy-3-methoxy-5-nitro-benzamide

To a suspension of 3-Methoxy-5-nitro-benzoic acid (2.957 g, 15 mmol) in dry dichloromethane (70 ml), oxalyl chloride (2.62 ml, 30 mmol) is added, followed by adding one drop of DMF. The mixture is stirred at room temperature for 2 hours, resulting a clear solution. The solvents are removed. The residue is dissolved in dichloromethane (70 ml), and O-ethylhydroxylamine hydrochloride (1.56 g, 16 mmol) is added. The mixture is cooled with ice-water bath and triethylamine (6.27 ml, 45 mmol) is added. The reaction mixture is allowed to warm up to room temperature, resulting a clean reaction in less than 1 hour. The reaction is quenched with saturated sodium bicarbonate aqueous solution. The organic layer is separated and washed by saturated sodium chloride solution and dried by Na₂SO₄. After removing the solvent, the crude product is purified by flash chromatography using EA/Hexane (50:50) as a white solid (3.42 g, 95%); ¹H NMR 400 MHz (CDCl₃) δ 8.94 (br, 1H), 8.12 (s, 1H), 7.85 (t, 1H, J=2.2 Hz), 7.67 (m, 1H), 4.13 (q, 2H, J=7.0 Hz), 3.93 (s, 3H), 1.35 (t, 3H, J=7.0 Hz); MS m/z 241.2 (M+1).

### Example 5d

### Preparation of 3-Amino-N-ethoxy-5-methoxy-benzamide

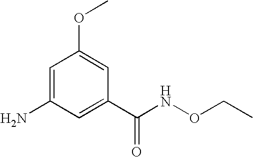

### 3-Amino-N-ethoxy-5-methoxy-benzamide

To a solution of N-Ethoxy-3-methoxy-5-nitro-benzamide (3.12 g, 13 mmol) in methanol (40 ml) is added Pd/C (100 mg). This mixture is charged with a hydrogen balloon. The reaction progress is monitored by TLC carefully. After the completion of the reaction, Pd/C is filtered off and the filtrate is concentrated under pressure to afford 3-Amino-N-ethoxy-5-methoxy-benzamide as a colorless oil (2.46 g, 90%); ¹H NMR 400 MHz (CDCl₃) δ 8.53 (br, 1H), 7.19 (s, 1H), 6.54 (m, 2H), 6.27 (m, 1H), 4.00 (q, 2H, J=7.0 Hz), 3.71 (s, 3H), 3.41 (s, 1H), 1.25 (t, 3H, J=7.0 Hz); MS m/z 211.2 (M+1).

### Example 5e

### Preparation of 3-[(2,4-Dichloro-pyrimidin-5-ylmethyl)-amino]-N-ethoxy-5-methoxy-benzamide

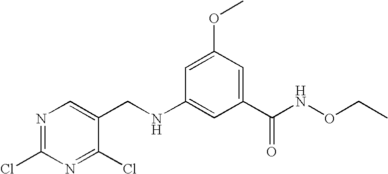

### 3-[(2,4-Dichloro-pyrimidin-5-ylmethyl)-amino]-N-ethoxy-5-methoxy-benzamide

3-Amino-N-ethoxy-5-methoxy-benzamide (2.31 g, 11 mmol) is added into a flask containing toluene (35 ml) and acetonitrile (5 ml), followed by adding sodium hydroxide (440 mg in 1.6 ml water, 11 mmol). Then a solution of 2,4-Dichloro-5-iodomethyl-pyrimidine (2.89 g, 10 mmol) in toluene (5 ml) and acetonitrile (5 ml) is slowly added. After the completion of the addition, the reaction mixture is stirred for 30 min at room temperature. After removal of all the solvents under pressure, the residue is dissolved in ethyl acetate and saturated sodium bicarbonate aqueous solution. The organic layer is separated and washed by saturated sodium chloride solution and dried by Na₂SO₄. After removing the solvent, the crude product is purified by flash chromatography using EA/Hexane (60:40) as a white solid (2.2 g, 59%); ¹H NMR 400 MHz (CDCl₃) δ 8.90 (br, 1H), 8.60 (s, 1H), 6.77 (s, 1H), 6.71 (s, 1H), 6.33 (s, 1H), 4.48 (s, 2H), 4.07 (q, 2H, J=7.0 Hz), 3.77 (s, 3H), 1.30 (t, 3H, J=7.0 Hz); MS m/z 371.2 (M+1).

### Example 5f

### Preparation of 3-[(2-Chloro-4-ethylamino-pyrimidin-5-ylmethyl)-amino]-N-ethoxy-5-methoxy-benzamide

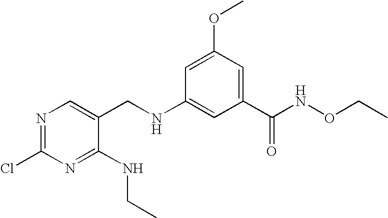

### 3-[(2-Chloro-4-ethylamino-pyrimidin-5-ylmethyl)-amino]-N-ethoxy-5-methoxy-benzamide

A solution of 3-[(2,4-Dichloro-pyrimidin-5-ylmethyl)-amino]-N-ethoxy-5-methoxy-benzamide (1.78 g, 4.8 mmol) in THF (15 ml) is cooled with ice-water bath, then ethylamine (1 ml 70% in water, 18 mmol) is added. The reaction mixture is kept at 0° C. for 1 hour. After removal of the solvents under pressure, the residue is dissolved in ethyl acetate and saturated sodium bicarbonate aqueous solution. The organic layer is separated and washed by saturated sodium chloride solution and dried by Na₂SO₄. After removing the solvent, the crude product is purified by flash chromatography using EA/Hexane (70:30) as a white form (1.5 g, 82%); ¹H NMR 400 MHz (CDCl₃) δ 9.59 (br, 1H), 7.78 (s, 1H), 6.70 (s, 1H), 6.66 (s, 1H), 6.38 (br, 1H), 6.30 (s, 1H), 4.07-4.03 (m, 4H), 3.75 (s, 3H), 3.51 (m, 2H), 1.28 (t, 3H, J=7.0 Hz), 1.21 (t, 3H, J=7.0 Hz); MS m/z 380.2 (M+1).

### Example 5g

### Preparation of 3-(7-Chloro-1-ethyl-2-oxo-1,4-dihydro-2H-pyrimido[4,5-d]pyrimidin-3-yl)-N-ethoxy-5-methoxy-benzamide

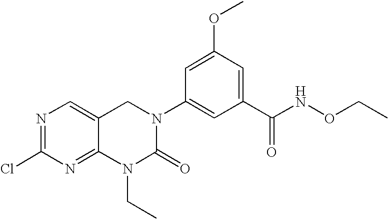

### 3-(7-Chloro-1-ethyl-2-oxo-1,4-dihydro-2H-pyrimido[4,5-d]pyrimidin-3-yl)-N-ethoxy-5-methoxy-benzamide

A solution of 3-[(2-Chloro-4-ethylamino-pyrimidin-5-ylmethyl)-amino]-N-ethoxy-5-methoxy-benzamide (531 mg, 1.4 mmol) and N,N-diisopropylethylamine (1.22 ml, 7 mmol) in THF (14 ml) is cooled with ice-water bath, then phenyl chloroformate (0.2 ml, 1.6 mmol) is added. The reaction is allowed to warm to room temperature for 1 hour. Then NaHMDS (2 ml 1M in THF, 2 mmol) is slowly added. The reaction mixture is stirred overnight. The reaction mixture is diluted in ethyl acetate and washed with saturated sodium bicarbonate aqueous solution. The organic layer is separated and washed by saturated sodium chloride solution and dried by Na₂SO₄. After removing the solvent, the crude product is purified by flash chromatography using ethyl acetate as a white form (300 mg, 74%); MS m/z 406.2 (M+1).

### Example 5h

### Preparation of 3-(7-Cyclopropylamino-1-ethyl-2-oxo-1,4-dihydro-2H-pyrimido[4,5-d]pyrimidin-3-yl)-N-ethoxy-5-methoxy-benzamide

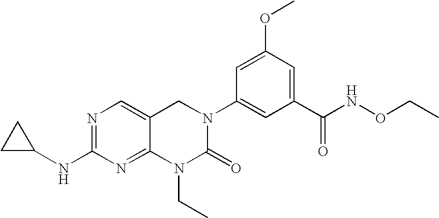

### 3-(7-Cyclopropylamino-1-ethyl-2-oxo-1,4-dihydro-2H-pyrimido[4,5-d]pyrimidin-3-yl)-N-ethoxy-5-methoxy-benzamide

A mixture of 3-(7-Chloro-1-ethyl-2-oxo-1,4-dihydro-2H-pyrimido[4,5-d]pyrimidin-3-yl)-N-ethoxy-5-methoxy-benzamide (20.3 mg, 0.05 mmol) in cyclopropyl amine (0.2 ml) is heated at 70° C. The reaction is complete in 5 hours. The final compound is purified by LCMS to afford the TFA salt of 3-(7-Cyclopropylamino-1-ethyl-2-oxo-1,4-dihydro-2H-pyrimido[4,5-d]pyrimidin-3-yl)-N-ethoxy-5-methoxy-benzamide as a white form (21.6 mg, 80%); ¹H NMR 400 MHz (CDCl₃) δ 11.51 (br, 1H), 7.86 (s, 1H), 7.16 (m, 1H), 7.04 (m, 1H), 6.96 (m, 1H), 4.53 (s, 2H), 3.81 (q, 2H, J=7.0 Hz), 3.80 (br, 1H), 3.74 (q, 2H, J=7.0 Hz), 2.50 (m, 1H), 1.02 (t, 3H, J=7.0 Hz), 0.61 (m, 2H), 0.41 (m, 2H); MS m/z 427.2 (M+1).

### Example 6

### Representative Compounds

By repeating the procedures described in the above examples, using appropriate starting materials, the following compounds of Formula (I), (II), or (III) are obtained (see Table 1).

TABLE 1
Representative compounds of Formula (I), (II), or (III)

| | | Physical Data |
|---|---|---|
| Com- | | ¹H NMR 400 MHz |
| pound | | (DMSO-d₆) and/or |
| Number | Structure | MS (m/z) |
| 1 | 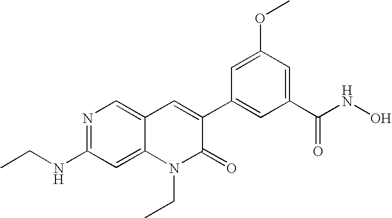 | ¹H NMR 400 MHz (DMSO- d₆) δ 11.32 (s, 1H), 8.55 (s, 1H), 8.12 (s, 1H), 7.69 (s, 1H), 7.47 (s, 1H), 7.34 (s, 1H), 6.47 (s, 1H), 4.23 (q, 2H, J = 7.2 Hz), 3.89 (s, 3H), 3.45 (q, 2H, J = 7.2 Hz), 3.24 (s, 1H), 1.31 (m, 6H); MS m/z 383.2 (M + 1). |
| 2 | 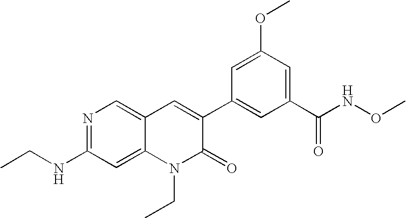 | ¹H NMR 400 MHz (DMSO- d₆) δ 11.99 (s, 1H), 8.70 (s, 1H), 8.27 (s, 1H), 7.82 (s, 1H), 7.63 (s, 1H), 7.47 (s, 1H), 6.62 (s, 1H), 4.38 (q, 2H, J = 7.2 Hz), 4.03 (s, 3H), 3.92 (s, 3H), 3.58 (q, 2H, J = 7.2 Hz), 3.37 (s, 1H), 1.42 (m, 6H); MS m/z 397.2 (M + 1). |
| 3 | 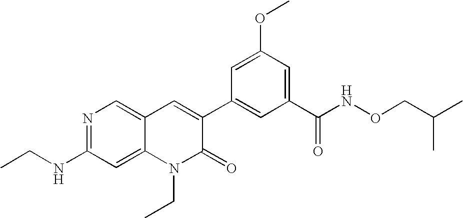 | MS m/z 439.2 (M + 1). |
| 4 | 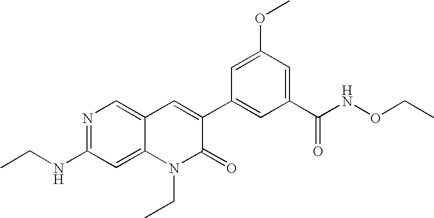 | MS m/z 411.2 (M + 1). |
| 5 | 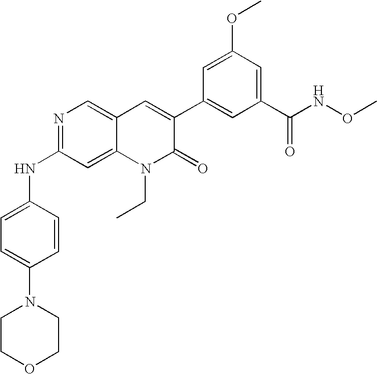 | ¹H NMR 400 MHz (DMSO- d₆) δ 11.74 (s, 1H), 9.37 (s, 1H), 8.51 (s, 1H), 8.05 (s, 1H), 7.58 (s, 1H), 7.46 (b, 2H), 7.40 (s, 1H), 7.21 (s, 1H), 6.97 (b, 2H), 6.59 (s, 1H), 4.11 (q, 2H, J = 7.2 Hz), 3.77 (s, 3H), 3.71 (b, 4H), 3.66 (s, 3H), 3.07 (b, 4H), 1.21 (t, 3H, J = 7.2 Hz); MS m/z 530.2 (M + 1). |
| 6 | 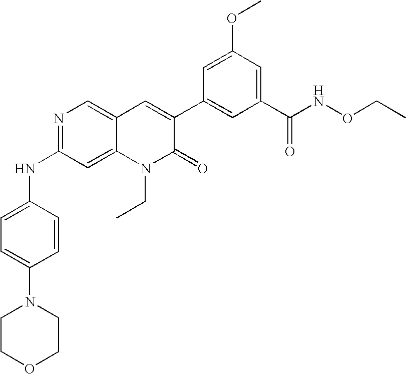 | ¹H NMR 400 MHz (DMSO- d₆) δ 11.60 (s, 1H), 9.37 (s, 1H), 8.51 (s, 1H), 8.07 (s, 1H), 7.58 (s, 1H), 7.44 (b, 2H), 7.39 (s, 1H), 7.21 (s, 1H), 6.99 (b, 2H), 6.58 (s, 1H), 4.15 (q, 2H, J = 7.2 Hz), 3.86 (q, 2H, J = 7.2 Hz), 3.77 (s, 3H), 3.71 (b, 4H), 3.66 (s, 3H), 3.07 (b, 4H), 1.17 (m, 6H); MS m/z 544.2 (M + 1). |
| 7 | 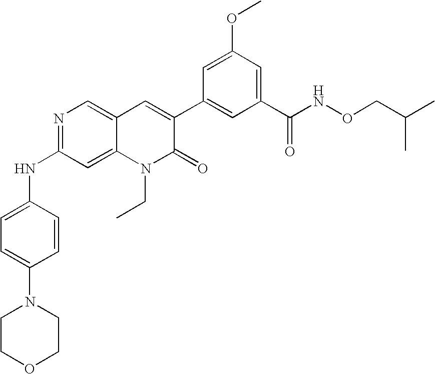 | MS m/z 572.2 (M + 1). |
| 8 | 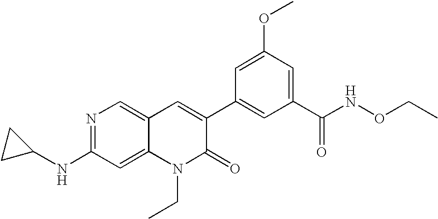 | MS m/z 423.2 (M + 1). |
| 9 | 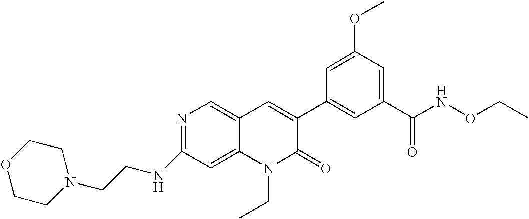 | MS m/z 496.2 (M + 1). |
| 10 | 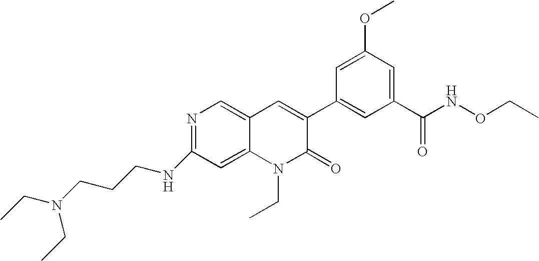 | MS m/z 496.2 (M + 1). |
| 11 | 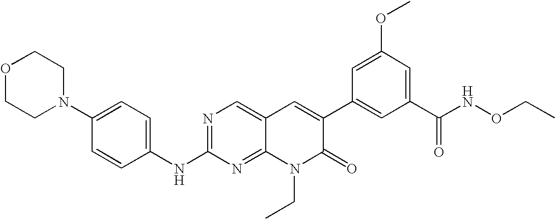 | ¹H NMR 400 MHz (DMSO- d₆) δ 11.69 (s, 1H), 9.98 (s, 1H), 8.80 (s, 1H), 8.08 (s, 1H), 7.69 (d, 2H, J = 8.8 Hz), 7.64 (s, 1H), 7.45 (s, 1H), 7.28 (s, 1H), 6.96 (d, 2H, J = 8.8 Hz), 4.40 (q, 2H, J = 6.8 Hz), 3.96 (q, 2H, J = 6.8 Hz), 3.84 (s, 3H), 3.75 (m, 4H), 3.08 (m, 4H), 1.30 (t, 3H, J = 6.8 Hz), 1.24 (t, 3H, J = 6.8 Hz); MS m/z 545.2 (M + 1). |
| 12 | 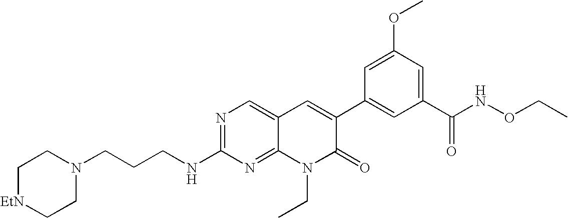 | MS m/z 538.3 (M + 1) |
| 13 | 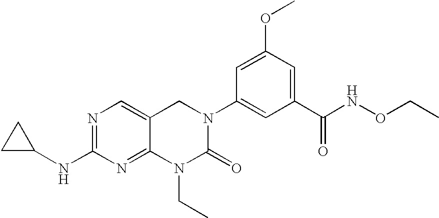 | ¹H NMR 400 MHz (CDCl₃) δ 11.51 (br, 1H), 7.86 (s, 1H), 7.16 (m, 1H), 7.04 (m, 1H), 6.96 (m, 1H), 4.53 (s, 2H), 3.81 (q, 2H,J = 7.0 Hz), 3.80 (br, 1H), 3.74 (q, 2H, J = 7.0 Hz), 2.50 (m, 1H), 1.02 (t, 3H, J = 7.0 Hz), 0.61 (m, 2H), 0.41 (m, 2H); MS m/z 427.2 (M + 1). |
| 14 | 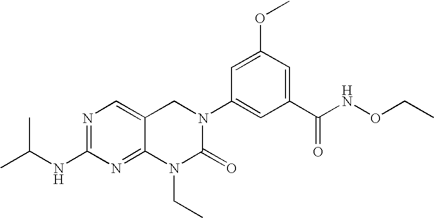 | ¹H NMR 400 MHz (CDCl₃) δ 11.63 (br, 1H), 8.10 (br, 1H), 7.95 (s, 1H), 7.28 (m, 1H), 7.17 (m, 1H), 7.08 (m, 1H), 4.63 (s, 2H), 3.93 (q, 2H, J = 7.0 Hz), 3.87 (q, 2H, J = 7.0 Hz), 2.40 (m, 1H), 1.15 (d, 3H, J = 7.0 Hz), 1.13 (d, 3H, J = 7.0 Hz); MS m/z 429.2 (M + 1). |
| 15 | 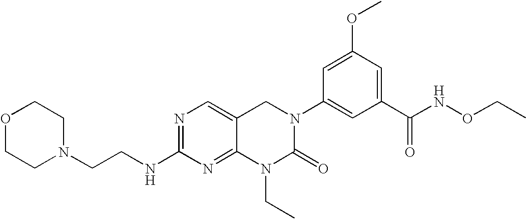 | MS m/z 500.3 (M + 1). |
| 16 | 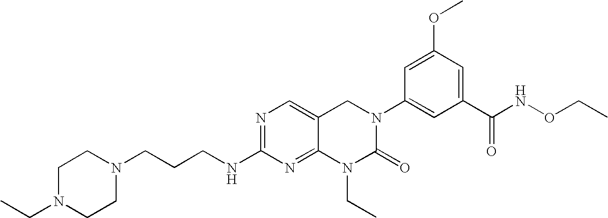 | MS m/z 541.3 (M + 1). |
| 17 | 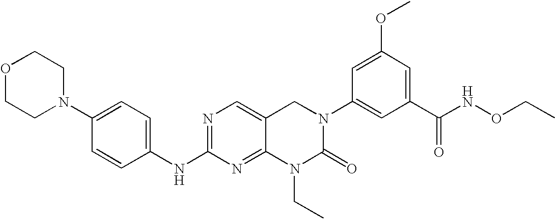 | MS m/z 548.3 (M + 1). |
| 18 | 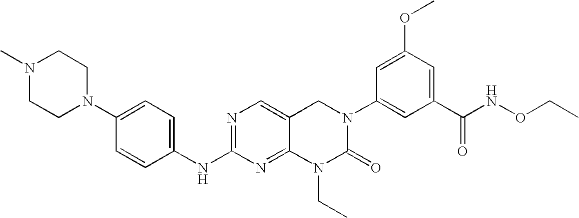 | MS m/z 561.3 (M + 1). |
| 19 | 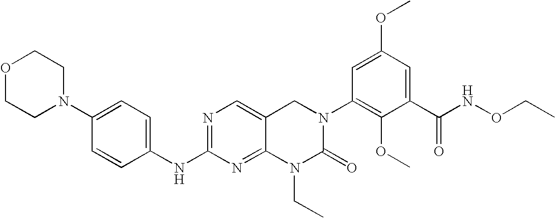 | MS m/z 578.2 (M + 1). |
| 20 | 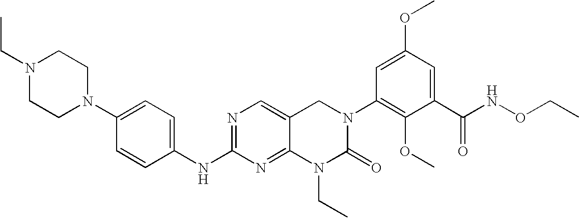 | MS m/z 605.3 (M + 1). |
| 21 | 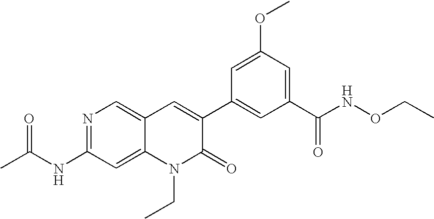 | MS m/z 425.2 (M + 1). |
| 22 | 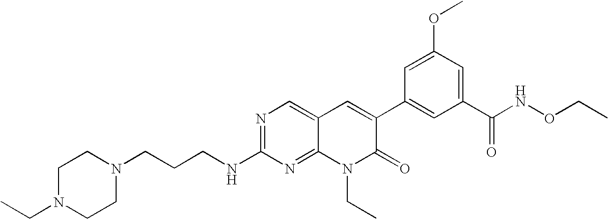 | |
| 23 | 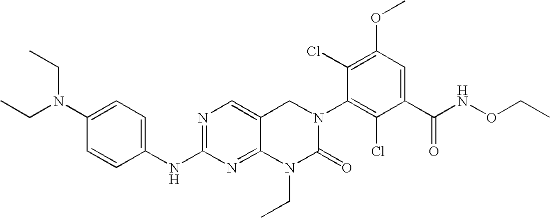 | MS m/z 603.5 (M + 1). |
| 24 | 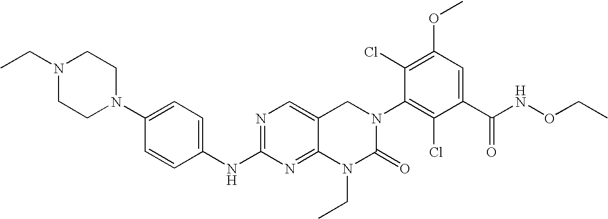 | MS m/z 644.5 (M + 1). |
| 25 | 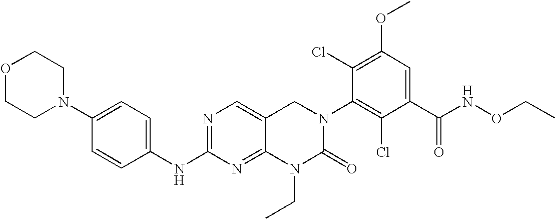 | MS m/z 617.5 (M + 1). |

Although it can be obvious for one ordinary skilled in the art, compounds having X₁═C and X₂═N corresponding to Formula (I), (II), or (III) can be synthesized using different starting materials as disclosed herein.

### Example 7

### Assays

Compounds of Formula (I), (II), or (III) are assayed to measure their capacity to selectively inhibit cell proliferation of 32D cells expressing BCR-Abl (32D-p210) compared with parental 32D cells. Compounds selectively inhibiting the proliferation of these BCR-Abl transformed cells are tested for anti-proliferative activity on Ba/F3 cells expressing either wild type or the mutant forms of Bcr-abl. In addition, compounds are assayed to measure their capacity to inhibit Abl, ALK, AMPK, Aurora, Axl, Bcr-Abl, BIK, Bmx, BRK, BTK, c-Kit, CSK, cSrc, CDK1, CHK2, CK1, CK2, CaMKII, CaMKIV, DYRK2, EGFR, EphB1, FES, FGFR1, FGFR2, FGFR3, Flt1, Flt3, FMS, Fyn, GSK3β, IGF-1R, IKKα, IKKβ, IR, IRAK4, ITK, JAK2, JAK3, JNK1α1, JNK2α, KDR, Lck, LYN, MAPK1, MAPKAP-K2, MEK1, MET, MKK4, MKK6, MST2, NEK2, NLK, p70S6K, PAK2, PDGFR, PDGFRα, PDK1, Pim-2, Plk3, PKA, PKBα, PKCα, PKCtheta, PKD2, c-Raf, RET, ROCK-I, ROCK-II, Ron, Ros, Rsk1, SAPK2a, SAPK2b, SAPK3, SAPK4, SGK, SIK, Syk, Tie2, TrkB, WNK3, and ZAP-70 kinases.

### Example 8

### Inhibition of Cellular BCR-Abl Dependent Proliferation (High Throughput Method)

The murine cell line used is the 32D hemopoietic progenitor cell line transformed with BCR-Abl cDNA (32D-p210). These cells are maintained in RPMI/10% fetal calf serum (RPMI/FCS) supplemented with penicillin 50 □g/ml, streptomycin 50 □g/ml and L-glutamine 200 mM. Untransformed 32D cells are similarly maintained with the addition of 15% of WEHI conditioned medium as a source of IL3.

50 □l of a 32D or 32D-p210 cells suspension are plated in Greiner 384 well microplates (black) at a density of 5000 cells per well. 50 nl of test compound (1 mM in DMSO stock solution) is added to each well (STI571 is included as a positive control). The cells are incubated for 72 hours at 37° C., 5% CO₂. 10 □l of a 60% Alamar Blue™ solution (Trek Diagnostics Systems, Inc., Westlake, Ohio) is added to each well and the cells are incubated for an additional 24 hours. The fluorescence intensity (Excitation at 530 nm, Emission at 580 nm) is quantified using the Acquest™ system (Molecular Devices Corp. Sunnyvale, Calif.).

### Example 9

### Inhibition of Cellular BCR-Abl Dependent Proliferation

32D-p210 cells are plated into 96 well TC plates at a density of 15,000 cells per well. 50 □L of two fold serial dilutions of the test compound (Cₘₐₓ is 40 □M) are added to each well (STI571 is included as a positive control). After incubating the cells for 48 hours at 37° C., 5% CO₂, 15 □L of MTT (Promega, Madison Wis.) is added to each well and the cells are incubated for an additional 5 hours. The optical density at 570 nm is quantified spectrophotometrically and IC₅₀ values, the concentration of compound required for 50% inhibition, determined from a dose response curve.

### Example 10

### Effect on Cell Cycle Distribution

32D and 32D-p210 cells are plated into 6 well TC plates at 2.5×10⁶ cells per well in 5 ml of medium and test compound at 1 or 10 □M is added (STI571 is included as a control). The cells are then incubated for 24 or 48 hours at 37° C., 5% CO₂. 2 ml of cell suspension is washed with PBS, fixed in 70% EtOH for 1 hour and treated with PBS/EDTA/RNase A for 30 minutes. Propidium iodide (Cf=10 □g/ml) is added and the fluorescence intensity is quantified by flow cytometry on the FACScalibur™ system (BD Biosciences, Rockville, Md.). Compounds of Formula (I), (II), or (III) demonstrate an apoptotic effect on the 32D-p210 cells but do not induce apoptosis in the 32D parental cells.

### Example 11

### Effect on Cellular BCR-Abl Autophosphorylation

BCR-Abl autophosphorylation is quantified with capture Elisa using a c-abl specific capture antibody and an antiphosphotyrosine antibody. 32D-p210 cells are plated in 96 well TC plates at 2×10⁵ cells per well in 50 □L of medium. 50 □L of two fold serial dilutions of test compounds (Cₘₐₓ is 10 □M) are added to each well (STI571 is included as a positive control). The cells are incubated for 90 minutes at 37° C., 5% CO₂. The cells are then treated for 1 hour on ice with 150 μL of lysis buffer (50 mM Tris-HCl, pH 7.4, 150 mM NaCl, 5 mM EDTA, 1 mM EGTA and 1% NP-40) containing protease and phosphatase inhibitors. 50 □L of cell lysate is added to 96 well optiplates previously coated with anti-abl specific antibody and blocked. The plates are incubated for 4 hours at 4° C. After washing with TBS-Tween 20 buffer, 50 □L of alkaline-phosphatase conjugated anti-phosphotyrosine antibody is added and the plate is further incubated overnight at 4° C. After washing with TBS-Tween 20 buffer, 90 □L of a luminescent substrate are added and the luminescence is quantified using the Acquest™ system (Molecular Devices Corp.). Compounds of Formula (I), (II), or (III) that inhibit the proliferation of the BCR-Abl expressing cells, inhibit the cellular BCR-Abl autophosphorylation in a dose-dependent manner.

### Example 12

### Effect on Proliferation of Cells Expressing Mutant Forms of Bcr-abl

Compounds of Formula (I), (II), or (III) are tested for their antiproliferative effect on Ba/F3 cells expressing either wild type or the mutant forms of BCR-Abl (G250E, E255V, T3151, F317L, M351T) that confers resistance or diminished sensitivity to STI571. The antiproliferative effect of these compounds on the mutant-BCR-Abl expressing cells and on the non transformed cells are tested at 10, 3.3, 1.1 and 0.37 □M as described above (in media lacking IL3). The IC₅₀ values of the compounds lacking toxicity on the untransformed cells are determined from the dose response curves obtained as describe above.

### Example 13

### b-Raf

Compounds of Formula (I), (II), or (III) are tested for their ability to inhibit the activity of b-Raf. The assay is carried out in 384-well MaxiSorp™ plates (NUNC, Rochester, N.Y.) with black walls and clear bottom. The substrate, I□B□F is diluted in DPBS (1:750) and 15 □l is added to each well. The plates are incubated at 4° C. overnight and washed 3 times with TBST (25 mM Tris, pH 8.0, 150 mM NaCl and 0.05% Tween-20) using the EMBLA plate washer (Molecular Devices). Plates are blocked by Superblock blocking buffer (Pierce Biotechnology, Inc. Rockford Ill.; 15 μl/well) for 3 hours at room temperature, washed 3 times with TBST and pat-dried. Assay buffer containing 20 □M ATP (10□l) is added to each well followed by 100 nl or 500 nl of compound. B-Raf is diluted in the assay buffer (1□l into 25 □l) and 10 □l of diluted b-Raf is added to each well (0.4 □g/well). The plates are incubated at room temperature for 2.5 hours. The kinase reaction is stopped by washing the plates 6 times with TBST. Phosph-I□B□Ser32/36) antibody is diluted in Superblock (1:10,000) and 15 □l is added to each well. The plates are incubated at 4° C. overnight and washed 6 times with TBST. AP-conjugated goat-anti-mouse IgG is diluted in Superblock (1:1,500) and 15 □l is added to each well. Plates are incubated at room temperature for 1 hour and washed 6 times with TBST. 15 □l of Attophos AP substrate is added to each well and plates are incubated at room temperature for 15 minutes. Plates are read on Acquest™ or AnalystGT™ (Molecular Devices Corp.) using a Fluorescence Intensity Nanxin BBT anion (505 dichroic mirror).

### Example 14

### FGFR3 (Enzymatic Assay)

Kinase activity assay with purified FGFR3 (Upstate) is carried out in a final volume of 10 μL containing 0.25 μg/ml of enzyme in kinase buffer (30 mM Tris-HCl pH7.5, 15 mM MgCl₂, 4.5 mM MnCl₂, 15 μM Na₃VO₄ and 50 μg/ml BSA), and substrates (5 μg/ml biotin-poly-EY (Glu, Tyr) (CIS-US, Inc.) and 3□M ATP). Two solutions are made: the first solution of 5 □l contains the FGFR3 enzyme in kinase buffer is first dispensed into 384-format Proxiplate® (Perkin-Elmer) followed by adding 50 nL of compounds dissolved in DMSO, then 5 □l of second solution containing the substrate (poly-EY) and ATP in kinase buffer is added to each well. Reactions are incubated at room temperature for one hour, stopped by adding 10 μL of HTRF detection mixture, which contains 30 mM Tris-HCl pH7.5, 0.5 M KF, 50 mM EDTA, 0.2 mg/ml BSA, 15 g/ml streptavidin-XL665 (CIS-US, Inc.) and 150 ng/ml cryptate conjugated anti-phosphotyrosine antibody (CIS-US, Inc.). After one hour of room temperature incubation to allow for streptavidin-biotin interaction, time resolved florescent signals are read on AnalystGT™ (Molecular Devices Corp.). IC₅₀ values are calculated by linear regression analysis of the percentage inhibition of each compound at 12 concentrations (1:3 dilution from 50 μM to 0.28 nM).

### Example 15

### FGFR3 (Cellular Assay)

Compounds of Formula (I), (II), or (III) are tested for their ability to inhibit transformed Ba/F3-TEL-FGFR3 cell proliferation, which is depended on FGFR3 cellular kinase activity. Ba/F3-TEL-FGFR3 are cultured up to 800,000 cells/ml in suspension, with RPMI 1640 supplemented with 10% fetal bovine serum as the culture medium. Cells are dispensed into 384-well format plate at 5000 cell/well in 50 μL culture medium. Compounds of Formula (I), (II), or (III) are dissolved and diluted in dimethylsufoxide (DMSO). Twelve points 1:3 serial dilutions are made into DMSO to create concentrations gradient ranging typically from 10 mM to 0.05 μM. Cells are added with 50 nL of diluted compounds and incubated for 48 hours in cell culture incubator. Alamar Blue™ (TREK Diagnostic Systems Inc.), which can be used to monitor the reducing environment created by proliferating cells, is added to cells at final concentration of 10%. After additional four hours of incubation in a 37° C. cell culture incubator, fluorescence signals from reduced Alamar Blue™ (Excitation at 530 nm, Emission at 580 nm) are quantified on AnalystGT™ (Molecular Devices Corp.). IC₅₀ values are calculated by linear regression analysis of the percentage inhibition of each compound at 12 concentrations.

### Example 16

### FLT3 (Cellular Assay) and Others

The effects of compounds of Formula (I), (II), or (III) on the cellular activity of FLT3 are conducted using identical methods as described above for FGFR3 cellular activity, except that Ba/F3-FLT3-ITD is used instead of Ba/F3-TEL-FGFR3. Similarly, other cell lines including, but not limited to, Ba/F3-TEL-ALK, Ba/F3-TEL-BMX, Ba/F3-TEL-EphB, Ba/F3-TEL-JAK2, Ba/F3-TEL-InsR, Ba/F3-TEL-LckB, Ba/F3-TEL-KitQ, Ba/F3-TEL-FGFR1, Ba/F3-TEL-SRC, or Ba/F3-TEL-PDGR, can be used for cellular assays.

### Example 17

### Upstate KinaseProfiler™—Radio-enzymatic Filter Binding Assay

Compounds of Formula (I), (II), or (III) are assessed for their ability to inhibit individual members of a panel of kinases (a partial, non-limiting list of kinases includes: Abl, ALK, AMPK, Aurora, Axl, Bcr-Abl, BIK, Bmx, BRK, BTK, c-Kit, CSK, cSrc, CDK1, CHK2, CK1, CK2, CaMKII, CaMKIV, DYRK2, EGFR, EphB1, FES, FGFR1, FGFR2, FGFR3, Flt1, Flt3, FMS, Fyn, GSK3β, IGF-1R, IKKα, IKKβ, IR, IRAK4, ITK, JAK2, JAK3, JNK1α1, JNK2α, KDR, Lck, LYN, MAPK1, MAPKAP-K2, MEK1, MET, MKK4, MKK6, MST2, NEK2, NLK, p70S6K, PAK2, PDGFR, PDGFRα, PDK1, Pim-2, Plk3, PKA, PKBα, PKCα, PKCtheta, PKD2, c-Raf, RET, ROCK-I, ROCK-II, Ron, Ros, Rsk1, SAPK2a, SAPK2b, SAPK3, SAPK4, SGK, SIK, Syk, Tie2, TrkB, WNK3, and ZAP-70). The compounds are tested in duplicates at a final concentration of 10 □M following this generic protocol. Note that the kinase buffer composition and the substrates vary for the different kinases included in the Upstate KinaseProfiler™ (Upstate Group LLC, Charlottesville, Va.) panel. The compounds are tested in duplicates at a final concentration of 10 □M following this generic protocol. Note that the kinase buffer composition and the substrates vary for the different kinases included in the Upstate KinaseProfiler™ panel (Upstate Group LLC). Kinase buffer (2.5 □L, 10×—containing MnCl₂ when required), active kinase (0.001-0.01 Units; 2.5 □L), specific or Poly(Glu-4-Tyr) peptide (5-500□M or 0.01 mg/ml) in kinase buffer and kinase buffer (50 □M; 5 □L) are mixed in an eppendorf on ice. A Mg/ATP mix (10 □L; 67.5 (or 33.75) mM MgCl₂, 450 (or 225) □M ATP and 1 □Ci/□l [□-³²P]-ATP (3000 Ci/mmol)) is added and the reaction is incubated at about 30° C. for about 10 minutes. The reaction mixture is spotted (20 □L) onto a 2 cm×2 cm P81 (phosphocellulose, for positively charged peptide substrates) or Whatman No. 1 (for Poly (Glu-4-Tyr) peptide substrate) paper square. The assay squares are washed 4 times, for 5 minutes each, with 0.75% phosphoric acid and washed once with acetone for 5 minutes. The assay squares are transferred to a scintillation vial, 5 ml scintillation cocktail are added and ³²P incorporation (cpm) to the peptide substrate is quantified with a Beckman scintillation counter. Percentage inhibition is calculated for each reaction.

Compounds of Formula (I), (II), or (III), in free form or in pharmaceutically acceptable derivative form, can exhibit valuable pharmacological properties, for example, as indicated by the in vitro tests described in this application. For example, compounds of Formula (I), (II), or (III) preferably show an IC₅₀ in the range of 1×10⁻¹⁰ to 1×10⁻⁵ M, preferably less than 50 nM for wild type BCR-Abl and G250E, E255V, T315I, F317L and M351T BCR-Abl mutants. Compounds of Formula (I), (II), or (III) preferably show an IC₅₀ in the range of 1×10⁻¹⁰ to 1×10⁻⁵ M, preferably less than 50 nM for FGFR3. Compounds of Formula (I), (II), or (III), at a concentration of 10 □M, preferably show a percentage inhibition of greater than 50%, preferably greater than about 70%, against Abl, BCR-Abl, Bmx, c-Raf, Csk, Fes, FGFR, Flt3, Ikk, IR, JNK, Lck, Mkk, PKC, PKD, Rsk, SAPK, Syk, Trk, BTK, Src, EGFR, IGF, Mek, Ros and Tie2 kinases.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1-46. (canceled)

47. A compound having the structure of Formula (III): 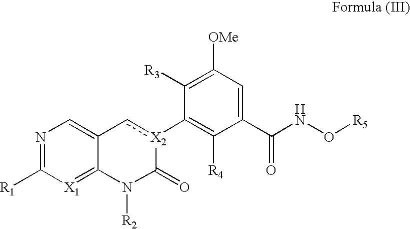
wherein:
R₁ is —H, —R′, —OR′, —NR′R″, —NR′″NR′R″, —NHCOR′, aliphatic amine, or aromatic amine, where R′ is selected from —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl; R″ is —H or C₁₋₈ alkyl, or R′ and R″ together with the nitrogen atom to form a C₃₋₁₀ heterocycloalkyl or C₅₋₁₀ heteroaryl; R″′ is a bond, C₁₋₆ alkylene, or arylene;
wherein any aryl, heteroaryl, cycloalkyl, and heterocycloalkyl of R′, R′″, or the combination of R′ and R″, is optionally substituted by one to three radicals independently selected from halo, hydroxy, nitro, cyano, C₁₋₆ alkyl optionally substituted with hydroxy, C₁₋₆ alkoxy, C₂₋₆ alkenyl, halo-substituted-C₁₋₆ alkyl, and halo-substituted-C₁₋₆ alkoxy;
R₂ is —H, —OH, halogen, optionally substituted C₁₋₆ alkyl, or optionally substituted C₁₋₆ alkoxy;
each of X₁ and X₂ is independently C or N;
each of R₃ and R₄ is independently —H, —CH₃, halogen, or alkoxyl;
R₅ is —H or optionally substituted C₁₋₆ alkyl;
and a pharmaceutically acceptable salt, pharmaceutically acceptable N-oxide, pharmaceutically active metabolite, pharmaceutically acceptable prodrug, pharmaceutically acceptable solvate thereof.

48. The compound of claim 47, wherein X₁═X₂═N.

49. The compound of claim 47, wherein X₁ is N and X₂ is C.

50. The compound of claim 47, wherein X₁ is CH and X₂═C.

51. The compound of claim 47, wherein R₁ is —H, —R′, —OR′, —NR′R″, —NR′″NR′R″, or —NHCOR′, where R′ is selected from —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl; R″ is —H or C₁₋₈ alkyl, or R′ and R″ together with the nitrogen atom to form a C₃₋₁₀ heterocycloalkyl or C₅₋₁₀ heteroaryl; R′″ is a bond, C₁₋₆ alkylene, or arylene.

52. The compound of claim 47, wherein R₁ is —H, —R′, —OR′, —NHCOR′, aliphatic amine, or aromatic amine, where R′ is selected from —H, C₁₋₆ alkyl, C₂₋₆ alkenyl, C₇₋₁₀ aryl-C₀₋₄ alkyl, C₅₋₁₀ heteroaryl-C₀₋₄ alkyl, C₃₋₁₀ cycloalkyl-C₀₋₄ alkyl, and C₃₋₁₀ heterocycloalkyl-C₀₋₄ alkyl.

53. The compound of claim 47, wherein R₁ is selected from the group consisting of

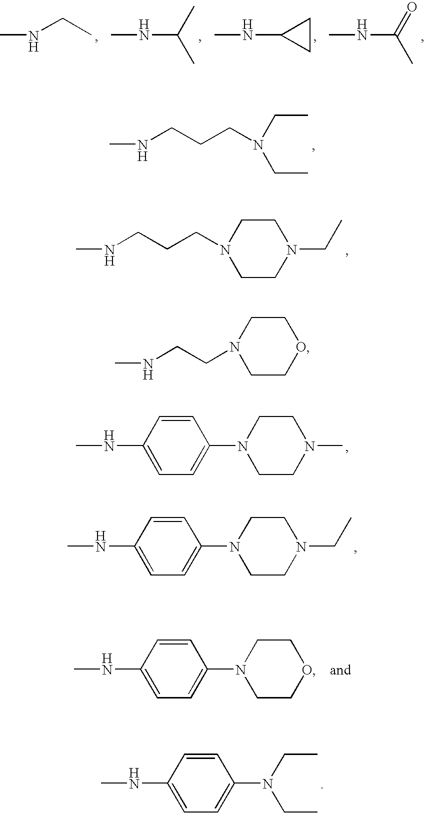

54. The compound of claim 47, wherein R₂ is —H or C₁₋₆ alkyl.

55. The compound of claim 47, wherein R₃ is —H or —CH₃.

56. The compound of claim 47, wherein R₄ is —H or —CH₃.

57. The compound of claim 47, wherein R₅ is —H or C₁₋₆ alkyl.

58. The compound of claim 47, selected from the group consisting of:

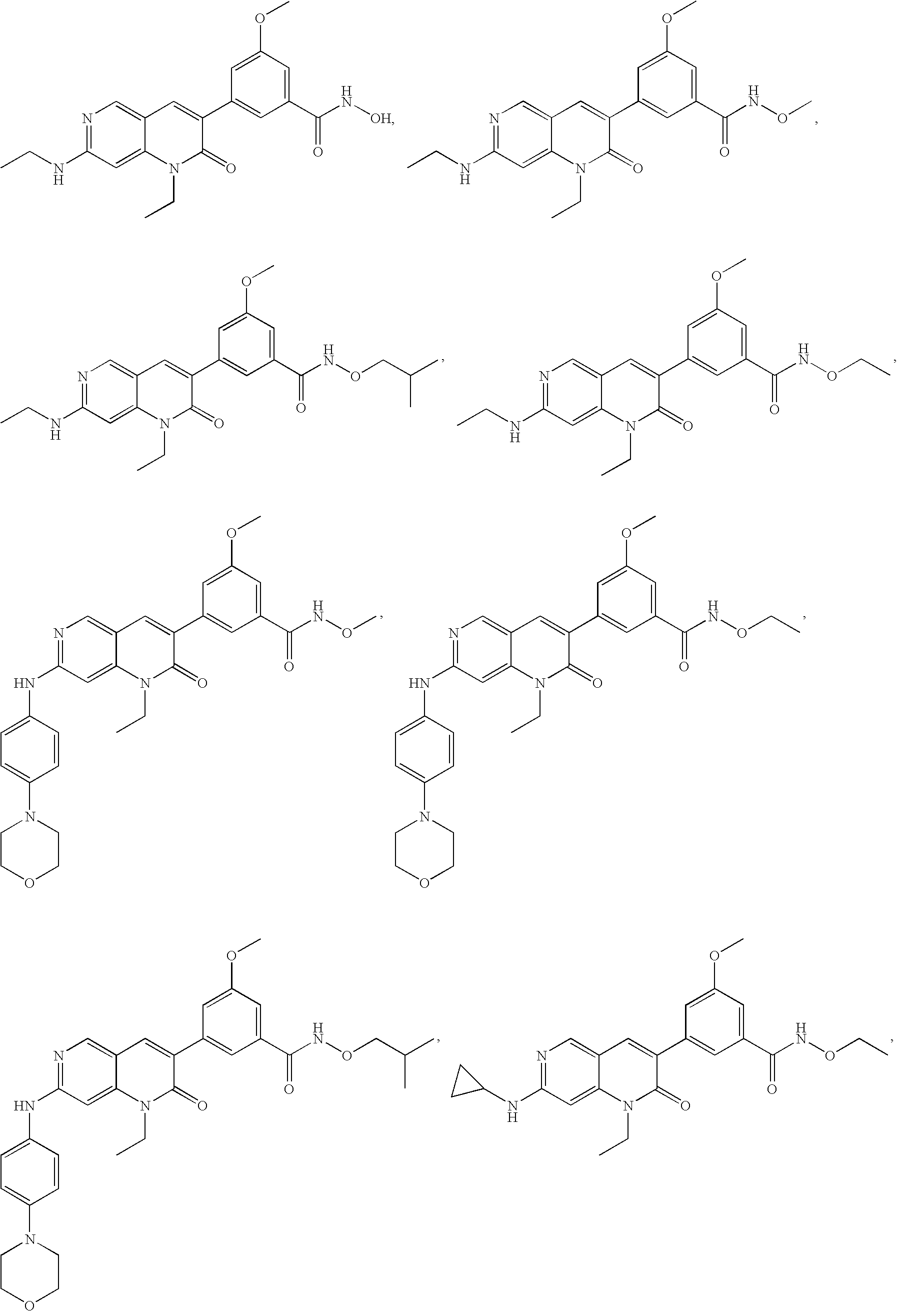

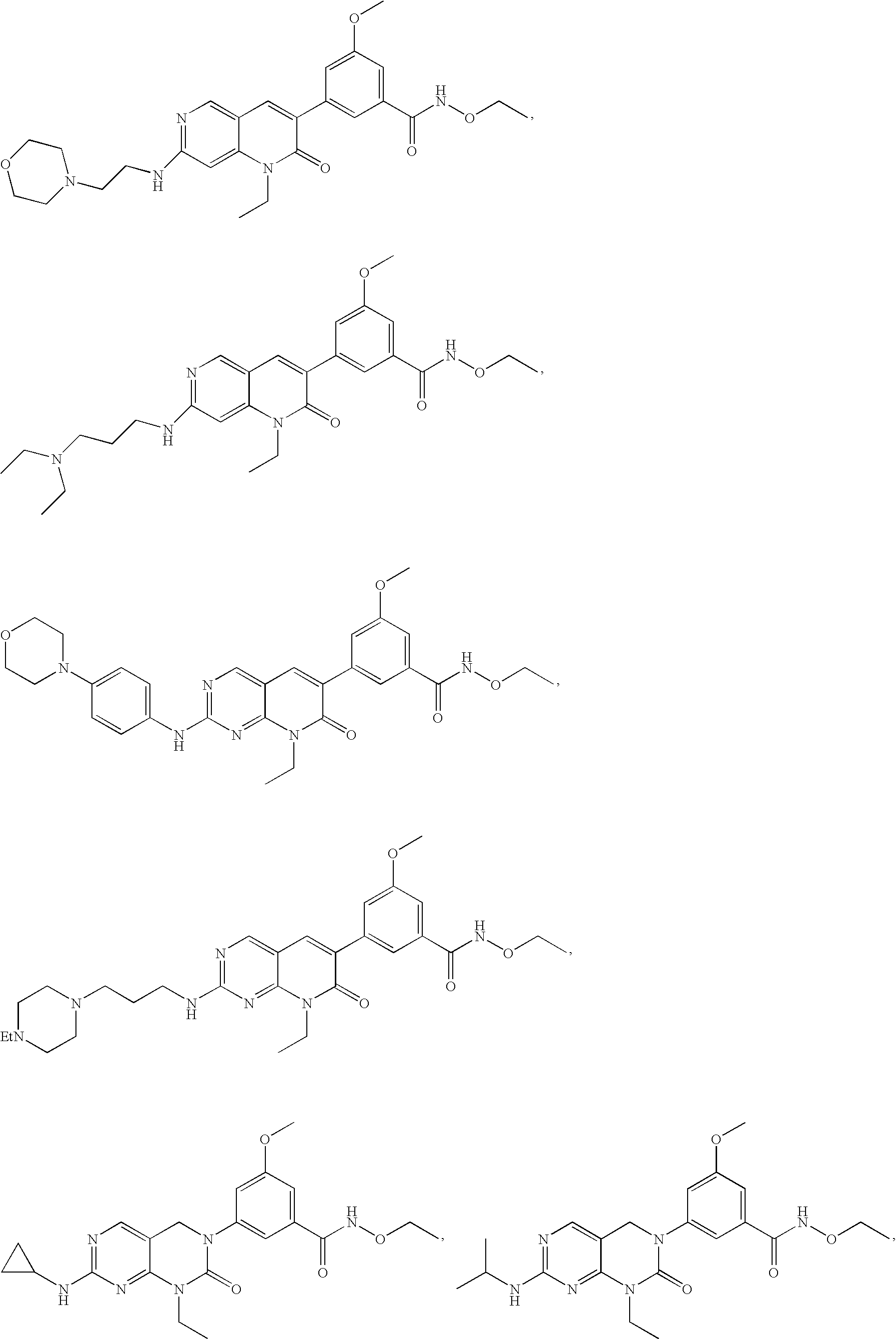

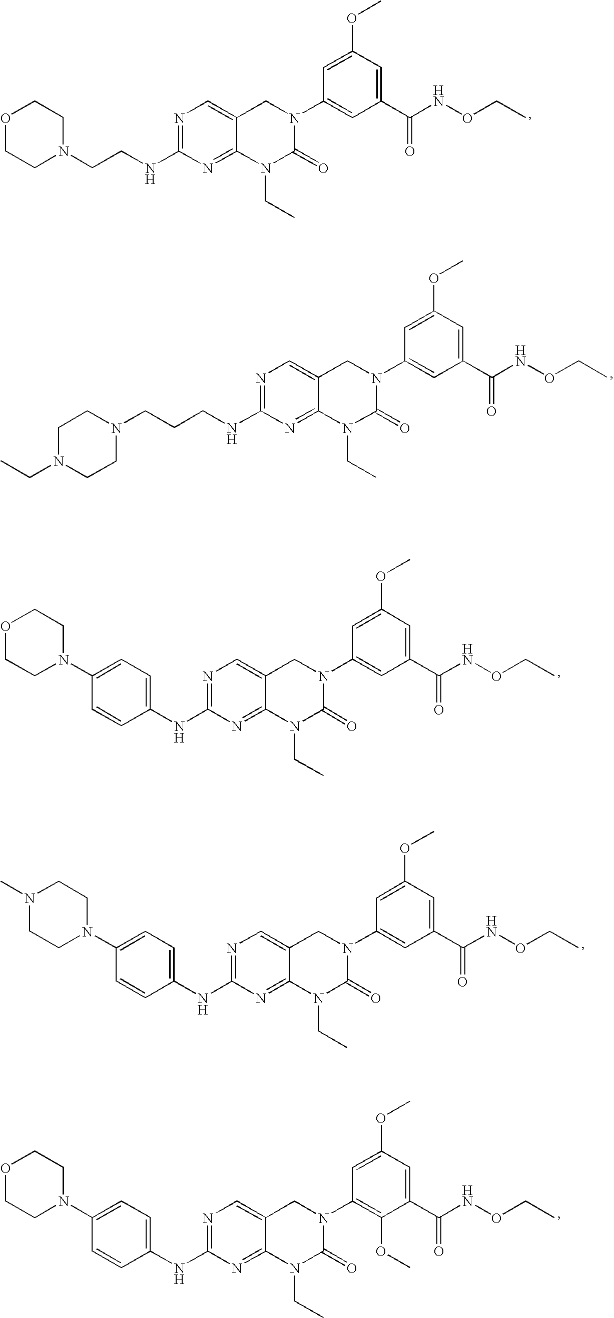

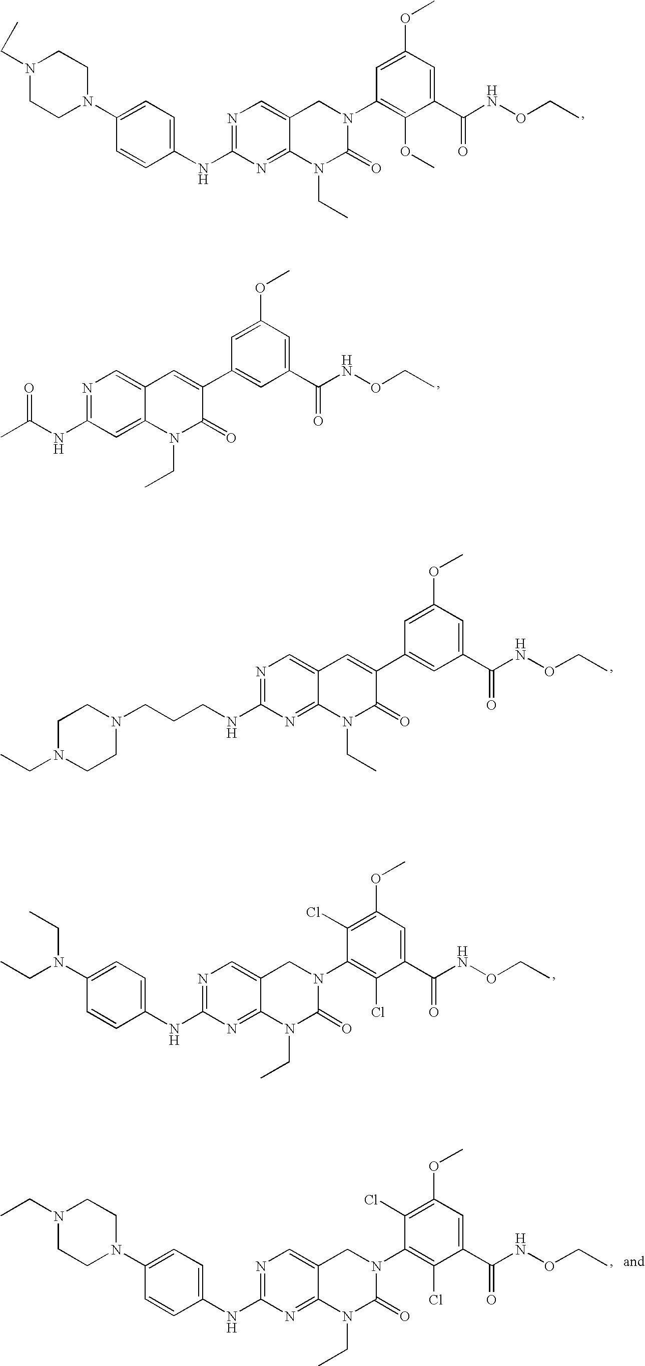

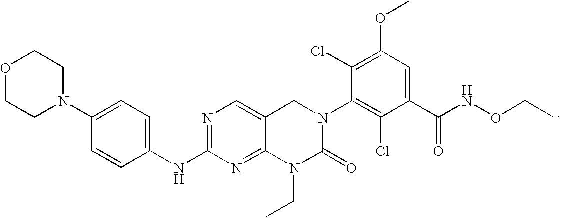

59. A pharmaceutical composition comprising a therapeutically effective amount of at least one compound of Formula (III) of claim 47, their respective N-oxide or other pharmaceutically acceptable derivatives, or individual isomers and mixtures of isomers thereof, in admixture with at least one pharmaceutically acceptable excipient.

60. A method of treating a disease in an animal in which inhibition of kinase activity can prevent, inhibit or ameliorate the pathology and/or symptomology of the disease, which method comprises administering to the animal a therapeutically effective amount of at least one compound of Formula (III) of claim 47, their respective N-oxide or other pharmaceutically acceptable derivatives, or individual isomers and mixtures of isomers thereof.

61. The method of claim 58, wherein the kinase is selected from the group consisting of Abl, ALK, AMPK, Aurora, Axl, Bcr-Abl, BIK, Bmx, BRK, BTK, c-Kit, CSK, cSrc, CDK1, CHK2, CK1, CK2, CaMKII, CaMKIV, DYRK2, EGFR, EphB1, FES, FGFR1, FGFR2, FGFR3, Flt1, Flt3, FMS, Fyn, GSK3β, IGF-1R, IKKα, IKKβ, IR, IRAK4, ITK, JAK2, JAK3, JNK1α1, JNK2α, KDR, Lck, LYN, MAPK1, MAPKAP-K2, MEK1, MET, MKK4, MKK6, MST2, NEK2, NLK, p70S6K, PAK2, PDGFR, PDGFRα, PDK1, Pim-2, Plk3, PKA, PKBα, PKCα, PKCtheta, PKD2, c-Raf, RET, ROCK-I, ROCK-II, Ron, Ros, Rsk1, SAPK2a, SAPK2b, SAPK3, SAPK4, SGK, SIK, Syk, Tie2, TrkB, WNK3, and ZAP-70.

62. The method of claim 58, wherein the kinase is selected from the group consisting of Abl, BCR-Abl, Bmx, c-Raf, Csk, Fes, FGFR, Flt3, Ikk, IR, JNK, Lck, Mkk, PKC, PKD, Rsk, SAPK, Syk, Trk, BTK, Src, EGFR, IGF, Mek, Ros and Tie2.

63-65. (canceled)

66. The method of claim 60, wherein the disease is selected from the group consisting of chronic myeloid leukemia (CML), acute lymphocytic leukemia, reimplantation of purified bone marrow cells, atherosclerosis, thrombosis, gliomas, sarcomas, prostate cancer, colon cancer, breast cancer, and ovary cancer, small cell lung cancer, psoriasis, scleroderma, fibrosis, protection of stem cells after treatment of chemotherapeutic agents, asthma, allogenic transplantation, tissue rejection, obliterative bronchiolitis (OB), restenosis, Wilms tumors, neuroblastomas, mammary epithelial cancer cells, thanatophoric dysplasia, growth arrest, abnormal bone development, myeloma-type cancers, hypertension, diabetic retinopathy, psoriasis, Kaposi's sarcoma, chronic neovascularization due to macular degeneration, rheumatoid arthritis, infantile haemangioma, rheumatoid arthritis, other autoimmune diseases, thrombin-induced platelet aggregation, immunodeficiency disorders, allergies, osteoporosis, osteoarthritis, neurodegenerative diseases, hepatic ischemia, myocardial infarction, congestive heart failure, other heart diseases, HTLV-1 mediated tumorigenesis, hyperplasia, pulmonary fibrosis, angiogenesis, stenosis, endotoxin shock, glomerular nephritis, genotoxic insults, chronic inflammation, and other inflammatory diseases.

67-69. (canceled)

70. The compound of claim 47, wherein each of R₃ and R₄ is independently —H or halogen.
